(19) United States
(12) Patent Application Publication
(10) Pub. No.: US 2026/0085311 A1
(43) Pub. Date: Mar. 26, 2026

(54) COMPOSITIONS AND METHODS FOR REDUCING RNA LEVELS

(71) Applicant: The Regents of the University of California, Oakland, CA (US)

(72) Inventors: Liana Lareau, Berkeley, CA (US); Fyodor Urnov, Richmond, CA (US); Ma. Carmelle Catamura, Berkeley, CA (US)

(21) Appl. No.: 18/876,098

(22) PCT Filed: Jun. 20, 2023
(86) PCT No.: PCT/US2023/068732

§ 371 Date: Dec. 17, 2024

Related U.S. Application Data
(60) Provisional application No. 63/354,096, filed on Jun. 21, 2022.

(51) Int. Cl. C12N 15/113; A61K 38/00; A61K 48/00; C12N 9/12; C12N 9/22; C12N 9/78; C12N 15/52; C12N 15/63

(52) U.S. Cl. CPC C12N 15/113; A61K 38/00; A61K 48/00; C12N 9/1276; C12N 9/222; C12N 9/78; C12N 15/52; C12N 15/63; C12Y 207/07049; C12Y 305/04004; C12Y 305/04005; C12N 2310/20

(57) ABSTRACT

The present disclosure provides methods for reducing the level of an RNA transcript from a target nucleic acid. The present disclosure provides methods of treating a disease that results from or is caused by a toxic gain-of-function protein. The present disclosure provides systems and compositions for carrying out a method of the present disclosure.

### CROSS-REFERENCE

This application claims the benefit of U.S. Provisional Patent Application No. 63/354,096, filed Jun. 21, 2022, which application is incorporated herein by reference in its entirety.

### INCORPORATION-BY-REFERENCE OF MATERIAL ELECTRONICALLY SUBMITTED

A Sequence Listing is provided herewith as a Sequence Listing XML, “BERK-473_SEQ_LIST.xml” created on Nov. 12, 2025 and having a size of 102,938 bytes. The contents of the Sequence Listing XML are incorporated by reference herein in their entirety.

### INTRODUCTION

Human diseases that follow a dominant negative inheritance pattern present a great challenge for treatment using gene therapy methods. In such cases, a copy of an allele is inherited from each parent: one is a pathogenic allele causing a disease phenotype (e.g., by exerting a toxic gain-of-function effect) and the other is a wild-type (non-pathogenic) allele. Allele-specific targeting is especially important when the wild-type allele is crucial to normal function, e.g., the wild-type allele encodes a protein whose function is critical.

Clustered Regularly Interspaced Short Palindromic Repeats (CRISPR)-Cas systems comprise a CRISPR-associated (Cas) effector polypeptide and a guide nucleic acid. Such CRISPR-Cas systems can bind to and modify a targeted nucleic acid. The programmable nature of these CRISPR-Cas effector systems has facilitated their use as a versatile technology for use in, e.g., gene editing.

There is a need in the art for methods of allele-specific gene editing.

### SUMMARY

The present disclosure provides methods for reducing the level of an RNA transcript from a target nucleic acid. The present disclosure provides methods for reducing the level of an RNA transcript from a target nucleic acid in an allele-specific manner. The present disclosure provides methods of treating a disease that results from or is caused by a toxic gain-of-function protein. The present disclosure provides systems and compositions for carrying out a method of the present disclosure.

### BRIEF DESCRIPTION OF THE DRAWINGS

FIG. 1 schematically depicts nonsense-mediated mRNA decay (NMD). An mRNA transcript with a premature stop codon (right) is flagged by the NMD surveillance pathway as erroneous and is tagged for RNA degradation.

FIG. 2 schematically depicts allele-specific targeting by engineered splicing. The “good” allele (non-pathogenic allele; e.g., encoding a wild-type protein) is depicted in comparison to a “bad” allele (pathogenic allele; e.g., encoding a toxic, gain-of-function polypeptide). A single-nucleotide polymorphism (SNP) distinguishes the “good” allele from the “bad” allele. The SNP in the pathogenic allele is used as a target area for introducing a poison exon.

FIG. 3 schematically depicts use of a CRISPR-Cas guide RNA and a donor DNA to introduce a poison exon, or to introduce a mutation that gives rise to a poison exon, in a target nucleic acid. In this example, the target region is in an intron of the HTT gene.

FIG. 4 depicts data showing successful introduction of a poison exon into the HTT gene and production of a spliced mRNA product that contains a premature stop codon.

FIG. 5 depicts homology-directed repair (HDR) ImageJ analysis showing production of a spliced mRNA product that contains a premature stop codon and degradation of the spliced mRNA via NMD.

FIG. 6 schematically depicts allele-specific targeting and editing using a base editor (in this schematic, a fusion protein comprising a CRISPR-Cas effector polypeptide and a cytidine deaminase) (reference, variant, and edited alleles: SEQ ID NOs: 1-3, respectively).

FIG. 7 schematically depicts gene editing using a base editor.

FIG. 8 schematically depicts gene editing using prime editing.

FIG. 9 shows SNPs from haplotypes common in Huntington's disease (HD).

FIG. 10 schematically depicts use of the rs363080 SNP to crease a poison exon selectively in a pathogenic HTT allele.

FIG. 11 schematically depicts use of the rs363080 SNP to crease a poison exon selectively in a pathogenic HTT allele (SEQ ID NO:4). The SNP is near the 3′ end of the guide sequence (5′-ACCCAAAGAAAAAGAGAGAACAA-3′; SEQ ID NO:5; where the SNP is bold an underlined). The SNP is within the PAM (CAA). In this instance, a CRISPR-Cas effector polypeptide that recognizes an NAA proto-spacer adjacent motif (PAM) can be used.

FIG. 12 schematically depicts use of the CRISPR-Cas effector polypeptide and the guide RNA shown in FIG. 11 to introduce a new AG acceptor splice site selectively into a pathogenic HTT allele (unedited and edited alleles: SEQ ID NOs:4 and 6, respectively).

FIG. 13 schematically depicts the effect of the introduction of the new AG acceptor splice site (depicted in FIG. 12; SEQ ID NO:7). The new AG acceptor splice site is used by the spliceosome to splice to a sequence starting with a GT sequence, thus generating a frameshift-induced stop codon in a spliced mRNA product.

FIG. 14 depicts the design of a micro poison exon (SEQ ID NOs:8 and 70).

FIG. 15A-15L provide amino acid sequences of exemplary CRISPR-Cas effector polypeptides (SEQ ID NOs:9-20; respectively).

FIG. 16 provides an amino acid sequence of an exemplary reverse transcriptase polypeptide (SEQ ID NO:21).

FIG. 17 schematically depicts AG and GT splice acceptor and donor sites, and nucleotide sequences surrounding the AG and GT splice acceptor and donor sites.

FIG. 18 depicts data showing successful introduction of a poison exon into the HTT gene by homology-directed repair (HDR) near rs684459 and production of a spliced mRNA product that contains a premature stop codon. In this case, the HDR donor DNA was designed to mimic the predicted results of base editing. ImageJ analysis shows production of a spliced mRNA product that contains a premature stop codon and degradation of the spliced mRNA via NMD.

FIG. 19 depicts electrophoresis data confirming that HCT116 cells are heterozygous at SNP3 (rs684459).

FIG. 20 depicts sequencing analysis after treating HCT116 cells with a guide specific for the C allele (SEQ ID NO:72) or a guide specific for the T allele (SEQ ID NO:73) of rs684459 (SEQ ID NO:74). The SNP3PE_C_donor sequence (SEQ ID NO:73) is depicted above. Analysis shows allele selectivity of guides.

FIG. 21 depicts data showing successful allele-specific introduction of a poison exon into the HTT gene by homology-directed repair (HDR) near rs684459 and production of a spliced mRNA product that contains a premature stop codon. In this case, the HDR donor DNA was designed to mimic the predicted results of prime editing, and Cas9 was targeted to only one allele. ImageJ analysis shows production of a spliced mRNA product that contains a premature stop codon and degradation of the spliced mRNA via NMD.

FIG. 22 schematically depicts use of a CRISPR-Cas guide RNA (SEQ ID NO:75) and a donor DNA (SEQ ID NO:76) to introduce a poison exon, or to introduce a mutation that gives rise to a poison exon, in a target nucleic acid. In this example, the target region is in an intron of the HTT gene (SEQ ID NO:80). Also depicted are Forward and Reverse primers (SEQ ID NOs:77-78; respectively) for RT-PCR of spliced mRNA.

FIG. 23 schematically depicts use of a CRISPR-Cas guide RNA and a donor DNA (SEQ ID NO:73) to introduce a poison exon, or to introduce a mutation that gives rise to a poison exon, in a target nucleic acid, in an allele-specific manner. In this example, the target region is in an intron of the HTT gene (SEQ ID NO:80). Also depicted are Forward and Reverse primers (SEQ ID NOs:77-78; respectively) for RT-PCR of spliced mRNA.

FIG. 24A-24B schematically depicts use of a CRISPR-Cas guide RNA and a base editor or CRISPR-Cas guide RNA and a prime editor to introduce a poison exon, or to introduce a mutation that gives rise to a poison exon, in a target nucleic acid, respectively. In these examples, the target region is in an intron of the HTT gene (SEQ ID NOs:81-82; respectively).

### DEFINITIONS

“Heterologous,” as used herein in the context of a polypeptide, refers to an amino acid sequence that is not found in the native polypeptide. For example, a fusion CRISPR-Cas effector polypeptide comprises: a) a CRISPR-Cas effector polypeptide; and b) one or more heterologous polypeptides, where the heterologous polypeptide comprises an amino acid sequence from a protein other than a CRISPR-Cas effector polypeptide. “Heterologous,” as used herein in the context of a nucleic acid, refers to a nucleotide sequence that is not found in the native nucleic acid. As an example, in a guide nucleic acid, a heterologous guide nucleotide sequence (present in a targeting segment) that can hybridize with a target nucleotide sequence (target region) of a target nucleic acid is a nucleotide sequence that is not found in nature in a guide nucleic acid together with a binding segment that can bind to a CRISPR-Cas effector polypeptide. For example, in some cases, a heterologous target nucleotide sequence (present in a heterologous targeting segment) is from a different source than a binding nucleotide sequence (present in a binding segment) that can bind to a CRISPR-Cas effector polypeptide of the present disclosure. For example, a guide nucleic acid may comprise a guide nucleotide sequence (present in a targeting segment) that can hybridize with a target nucleotide sequence present in a eukaryotic target nucleic acid. A guide nucleic acid of the present disclosure can be generated by human intervention and can comprise a nucleotide sequence not found in a naturally-occurring guide nucleic acid.

The term “naturally-occurring” as used herein as applied to a nucleic acid, a protein, a cell, or an organism, refers to a nucleic acid, cell, protein, or organism that is found in nature.

The terms “polynucleotide” and “nucleic acid,” used interchangeably herein, refer to a polymeric form of nucleotides of any length, either ribonucleotides or deoxynucleotides or combinations thereof. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The terms “polynucleotide” and “nucleic acid” should be understood to include, as applicable to the embodiment being described, single-stranded (such as sense or antisense) and double-stranded polynucleotides. It should be noted that, where a nucleic acid is an RNA, in some cases, the sequence is provided with “T” nucleotides; those skilled in the art will understand that the “T” nucleotides are “U” nucleotides in an RNA molecule.

As used herein, the term “guide RNA” (gRNA) and the like refer to an RNA that guides a CRISPR-Cas effector polypeptide (or a fusion protein comprising a CRISPR-Cas effector polypeptide) to a target sequence in a target nucleic acid. The term gRNA can also refer to a prime editing guide RNA (pegRNA), a nicking guide RNA (ngRNA), and a single guide RNA (sgRNA). In some cases, the term “gRNA molecule” refers to a nucleic acid encoding a gRNA. In some cases, the gRNA molecule is naturally occurring. In some cases, a gRNA molecule is non-naturally occurring. In some cases, a gRNA molecule is a synthetic gRNA molecule.

The terms “polypeptide,” “peptide,” and “protein”, are used interchangeably herein, refer to a polymeric form of amino acids of any length, which can include genetically coded and non-genetically coded amino acids, chemically or biochemically modified or derivatized amino acids, and polypeptides having modified peptide backbones. The term includes fusion proteins, including, but not limited to, fusion proteins with a heterologous amino acid sequence.

Polypeptides as described herein also include polypeptides having various amino acid additions, deletions, or substitutions relative to the native amino acid sequence of a polypeptide of the present disclosure. In some embodiments, polypeptides that are homologs of a polypeptide of the present disclosure contain non-conservative changes of certain amino acids relative to the native sequence of a polypeptide of the present disclosure. In some embodiments, polypeptides that are homologs of a polypeptide of the present disclosure contain conservative changes of certain amino acids relative to the native sequence of a polypeptide of the present disclosure, and thus may be referred to as conservatively modified variants. A conservatively modified variant may include individual substitutions, deletions or additions to a polypeptide sequence which result in the substitution of an amino acid with a chemically similar amino acid. Conservative substitution tables providing functionally similar amino acids are well-known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). A modification of an amino acid to produce a chemically similar amino acid may be referred to as an analogous amino acid.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence similarity can be determined in a number of different manners. To determine sequence identity, sequences can be aligned using the methods and computer programs, including BLAST, available over the world wide web at ncbi.nlm.nih.gov/BLAST. See, e.g., Altschul et al. (1990), J. Mol. Biol. 215:403-10. Another alignment algorithm is FASTA, available in the Genetics Computing Group (GCG) package, from Madison, Wisconsin, USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, California, USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate expression of a coding sequence and/or production of an encoded polypeptide in a host cell.

The term “transformation” is used interchangeably herein with “genetic modification” and refers to a permanent or transient genetic change induced in a cell following introduction of new nucleic acid (e.g., DNA exogenous to the cell) into the cell. Genetic change (“modification”) can be accomplished either by incorporation of the new nucleic acid into the genome of the host cell, or by transient or stable maintenance of the new nucleic acid as an episomal element. Where the cell is a eukaryotic cell, a permanent genetic change is generally achieved by introduction of new DNA into the genome of the cell.

“Operably linked” refers to a juxtaposition wherein the components so described are in a relationship permitting them to function in their intended manner. For instance, a promoter is operably linked to a coding sequence if the promoter affects its transcription or expression. As used herein, the terms “heterologous promoter” and “heterologous control regions” refer to promoters and other control regions that are not normally associated with a particular nucleic acid in nature. For example, a “transcriptional control region heterologous to a coding region” is a transcriptional control region that is not normally associated with the coding region in nature.

By “base editor (BE)” or “nucleobase editor (NBE)” is meant an agent that binds a polynucleotide and has nucleobase modifying activity. In some cases, the base editor comprises a nucleobase-modifying polypeptide (e.g., a deaminase) and a CRISPR-Cas effector polypeptide in conjunction with a guide nucleic acid (e.g., guide RNA). In some cases, the agent is a biomolecular complex comprising a polypeptide having base editing activity, i.e., a domain capable of modifying a base (e.g., A, T, C, G, or U) within a nucleic acid molecule (e.g., DNA). In some cases, the polynucleotide CRISPR-Cas effector polypeptide is fused or linked to a deaminase polypeptide. In some cases, the agent is a fusion protein comprising a polypeptide having base editing activity. In some cases, the polypeptide having base editing activity is linked to the guide RNA (e.g., via an RNA binding motif on the guide RNA and an RNA binding polypeptide fused to the deaminase). In some cases, the polypeptide having base editing activity is capable of deaminating a nucleobase within a nucleic acid. In some cases, the base editor is capable of deaminating one or more bases within a DNA molecule. In some cases, the base editor is capable of deaminating an adenosine (A) within DNA. In some cases, the base editor is an adenosine base editor (ABE). In some cases, the base editor is capable of deaminating a cytosine (C) within DNA. In some cases, the base editor is a cytosine base editor (CBE).

By “base editing activity” is meant acting to chemically alter a base within a polynucleotide. In one embodiment, a first base is converted to a second base. In cases, the base editing activity is cytidine deaminase activity, e.g., converting a C G base pair to a T A base pair. In some cases, the base editing activity is adenosine or adenine deaminase activity, e.g., converting an A T base pair to a G C base pair. In some cases, the base editing activity is cytosine deaminase activity, e.g., converting a target C G base pair to a T A base pair and adenosine or adenine deaminase activity, e.g., converting an A T base pair to a G C base pair.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to an individual organism, e.g., a mammal, including, but not limited to, murines, simians, humans, non-human primates, ungulates, felines, canines, bovines, ovines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a guide RNA” includes a plurality of such guide RNAs and reference to “the CRISPR-Cas effector polypeptide” includes reference to one or more CRISPR-Cas effector polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed. No language in the specification should be construed as indicating any non-claimed element as essential to the practice of the embodiments of the disclosure.

As used herein, the term “about” used in connection with an amount indicates that the amount can vary by 10% of the stated amount. For example, “about 100” means an amount of from 90-110. Where “about” is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for reducing the level of an RNA transcript from a target nucleic acid. The present disclosure provides methods of treating a disease that results from production of a toxic gain-of-function protein. The present disclosure provides systems and compositions for carrying out a method of the present disclosure.

### Methods for Reducing the Level of an RNA Transcript

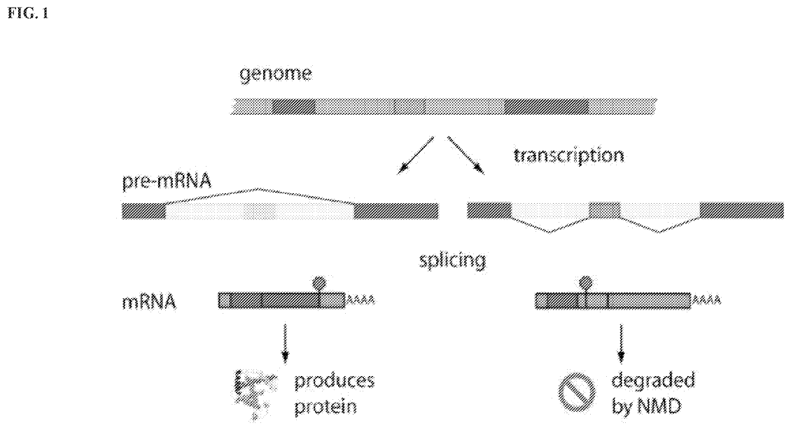

The present disclosure provides methods of reducing the level of an RNA transcript from a target nucleic acid (e.g., a target double-stranded DNA). In some cases, the methods comprise comprising modifying the nucleotide sequence of the target nucleic acid such that a spliced mRNA product of the modified target nucleic acid comprises a stop codon that is not present in a spliced mRNA transcript of the unmodified target nucleic acid (e.g., the target nucleic acid before it is modified), such that, as a result of the stop codon, the spliced mRNA product undergoes nonsense-mediated mRNA decay, thereby reducing the level of the RNA transcript. In some cases, the methods comprise comprising modifying the nucleotide sequence of the target nucleic acid such that a spliced mRNA product of the modified target nucleic acid comprises an exon that comprises a stop codon that is not present in a spliced mRNA transcript of the unmodified target nucleic acid, such that, as a result of the stop codon, the spliced mRNA product undergoes nonsense-mediated mRNA decay, thereby reducing the level of the RNA transcript. In some cases, a method of the present disclosure involves modifying a target nucleic acid so that a spliced mRNA product of the modified target nucleic acid includes a “poison exon”, i.e., an exon that introduces a premature stop codon (also referred to as a “premature termination codon” or “PTC”). The spliced mRNA product containing the premature stop codon may then be degraded by nonsense-mediated decay (NMD). NMD is a surveillance system that detects and degrades RNA transcripts that harbor PTCs. This is depicted schematically in FIG. 1.

The present disclosure provides a method of reducing the level of an RNA transcript from a target nucleic acid, the method comprising modifying the nucleotide sequence of the target nucleic acid such that a spliced mRNA product of the modified target nucleic acid comprises a stop codon that was not present in a spliced mRNA transcript of the target nucleic acid prior to the modification and wherein, as a result of the stop codon now present in the spliced mRNA product, the spliced mRNA product of the modified target nucleic acid undergoes nonsense-mediated mRNA decay, thereby reducing the level of the RNA transcript.

In some cases, the methods comprise modifying the nucleotide sequence of the target nucleic acid near a naturally-occurring intron sequence capable of acting as a poison exon if mis-spliced in the unmodified target, such that following modification according to the methods described herein, splicing of the RNA transcript of the modified target nucleic acid results in a spliced mRNA product that comprises an exon that comprises a stop codon that is not commonly present in a spliced mRNA transcript of the unmodified target nucleic acid, such that, as a result of the stop codon, the spliced mRNA product undergoes nonsense-mediated mRNA decay, thereby reducing the level of the RNA transcript.

By a reduction in the “level of an RNA transcript” is meant a reduction in the number of RNA transcripts, which can lead to a reduction in the level of the polypeptide encoded by the RNA transcripts. For example, the number of spliced mRNA products produced from the modified target nucleic acid is at least 5% lower than the number of spliced mRNA products produced from the unmodified target nucleic acid (the target nucleic acid prior to modification). For example, the number of spliced mRNA products produced from the modified target nucleic acid is at least 5%, at least 10%, at least 20%, at least 25%, at least 30%, at least 40%, at least 50%, at least 60%, at least 70%, at least 80%, or more than 80%, lower than the number of spliced mRNA products produced from the unmodified target nucleic acid (the target nucleic acid prior to modification).

In some cases, reduction in the number of RNA transcripts results in a reduction in the level of the protein encoded by the modified target nucleic acid. For example, the level of a protein encoded by the modified target nucleic acid in a cell that harbors the modified target nucleic acid is at least 5% lower (e.g., at least 5%, at least 10%, at least 20%, at least 25%, at least 30%, at least 40%, at least 50%, at least 60%, at least 70%, at least 80%, or more than 80%) than the level of the protein produced in a cell that harbors the unmodified target nucleic acid. Thus, the present disclosure provides a method of reducing the level of a toxic gain-of-function polypeptide produced by a cell, the method comprising modifying the nucleotide sequence of the target nucleic acid present in the cell, where the target nucleic acid encodes the toxic gain-of-function polypeptide, such that a spliced mRNA product of the modified target nucleic acid comprises a stop codon that was not present in a spliced mRNA transcript of the target nucleic acid prior to the modification and wherein, as a result of the stop codon now present in the spliced mRNA product, the spliced mRNA product of the modified target nucleic acid undergoes nonsense-mediated mRNA decay, thereby reducing the level of the RNA transcript and the encoded toxic gain-of-function polypeptide.

In some cases, modifying a target nucleic acid according to a method of the present disclosure comprises modifying the nucleotide sequence of the target nucleic acid that is within about 10 nucleotides of an AG dinucleotide in the target nucleic acid, such that the splicing machinery in a spliceosome in a cell would recognize the AG dinucleotide as a splice acceptor. For example, in some cases, modifying a target nucleic acid comprises modifying a polypyrimidine tract that is immediately 5′ of an endogenous AG dinucleotide, generating a modified AG dinucleotide-containing sequence, such that a spliceosome in a cell would recognize the modified AG dinucleotide-containing sequence as a splice acceptor dinucleotide. FIG. 17 provides a schematic depiction of nucleotide sequences surrounding an AG splice acceptor.

In some cases, modifying a target nucleic acid according to a method of the present disclosure comprises modifying a consensus sequence within 10 nucleotides of an endogenous GT dinucleotide, generating a modified GT dinucleotide-containing sequence, such that a spliceosome in a cell would recognize the modified GT dinucleotide-containing sequence as a splice donor dinucleotide. FIG. 17 provides a schematic depiction of nucleotide sequences surrounding a GT splice donor.

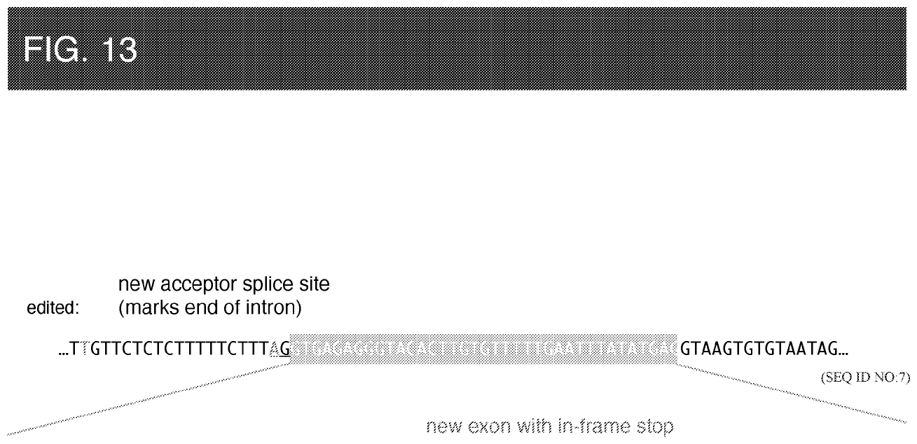

In some cases, modifying a target nucleic acid according to a method of the present disclosure comprises modifying a target sequence in the target nucleic acid to include a splice dinucleotide that is not present in the target nucleic acid, wherein the splice dinucleotide results in a spliced mRNA product that comprises a stop codon, optionally wherein the splice dinucleotide is an AG dinucleotide or a GT dinucleotide. In some cases, modifying a target nucleic acid according to a method of the present disclosure comprises modifying a target sequence in the target nucleic acid to include a splice dinucleotide that is not present in the target nucleic acid, wherein the splice dinucleotide results in a spliced mRNA product that comprises a frameshift-induced stop codon, optionally wherein the splice dinucleotide is an AG dinucleotide or a GT dinucleotide. See, e.g., FIG. 13 as an example, where a target nucleic acid is modified to include an AG acceptor splice site, generating a modified target nucleic acid with a non-naturally occurring AG acceptor splice site (e.g., the AG acceptor splice site occurs at a position that where, in the unmodified target nucleic acid, no such AG acceptor splice site occurs). When a pre-mRNA is synthesized using the modified target nucleic acid as a template, the splicing machinery in the spliceosome of a cell harboring the modified target nucleic acid can splice the sequence containing the non-naturally occurring AG acceptor splice site to a downstream GT sequence, generating a spliced mRNA that may include a frameshift-induced stop codon, i.e., a premature stop codon. The resulting spliced mRNA may be degraded via NMD.

In other cases, an exon of the target nucleic acid is modified such that the modified target nucleic acid includes a stop codon that is not present in the exon of the target nucleic acid.

Modification of a nucleotide sequence in a target nucleic acid can be carried out using any known method. For example, in some cases, the target nucleic acid is contacted with a CRISPR-Cas effector polypeptide and a guide nucleic acid (e.g., a guide RNA), wherein the guide nucleic acid comprises: i) a targeting region that comprises a nucleotide sequence that binds to a target sequence in the target nucleic acid; and ii) a protein-binding region that binds to the CRISPR-Cas effector polypeptide. As one example, the modification can be introduced by using a donor nucleic acid that comprises the desired nucleotide sequence modification. As another example, the modification can be introduced using a base editor. As another example, the modification can be introduced using a prime editor.

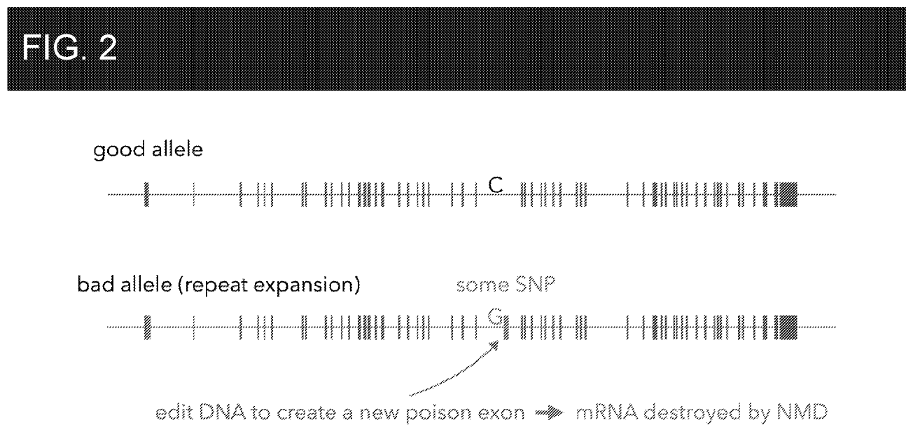

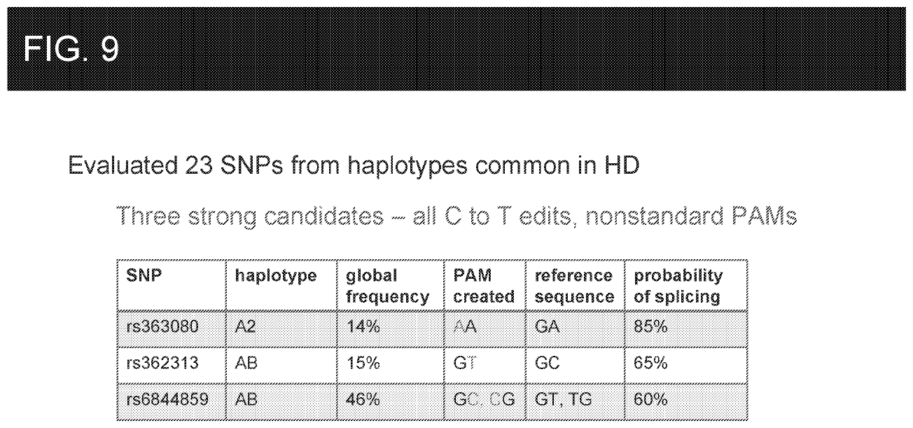

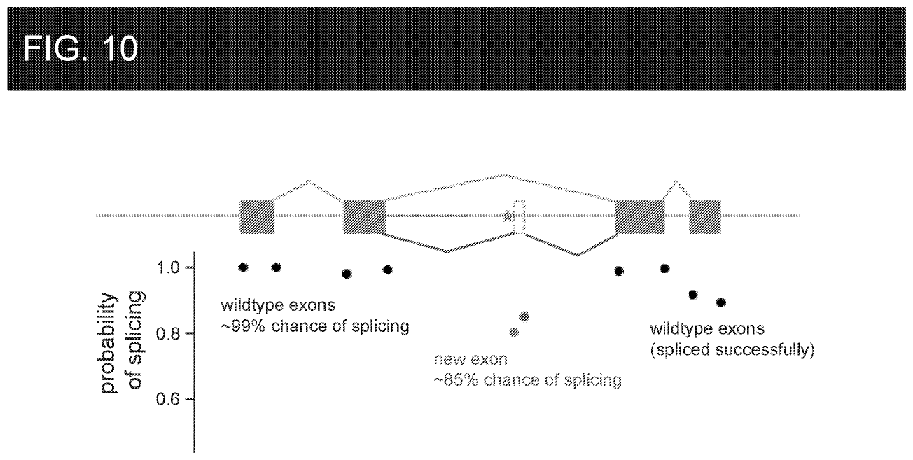

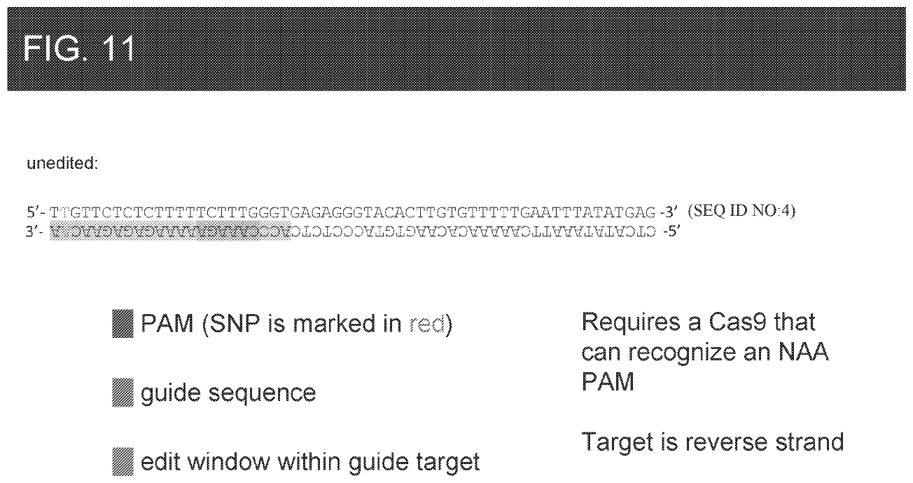

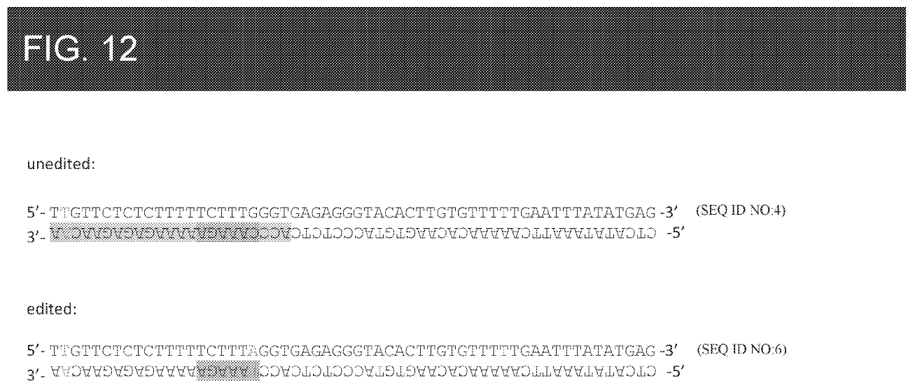

In some cases, the target nucleic acid is a double-stranded DNA. In some cases, the target nucleic acid is genomic DNA. In some cases, the target nucleic acid is present in a eukaryotic cell in vitro. In some cases, the target nucleic acid is present in a eukaryotic cell in vivo. In some cases, the eukaryotic cell comprises a disease-associated mutation-containing allele and a corresponding wild-type allele that does not comprise the disease-associated mutation, and the disease-associated mutation-containing allele comprises the target nucleic acid. In some cases, the present disclosure provides methods for allele-specific reduction of RNA, by introducing a poison exon (e.g., introducing a mutation that gives rise to a poison exon in a spliced RNA transcript of a modified target nucleic acid) into a target nucleic acid in first allele that comprises disease-associated mutation, but not into the corresponding wild-type allele. In some cases, this allele-specific modification is effected by using a guide RNA that comprises a target-binding region that binds to a target nucleotide in a target nucleic acid, where the target nucleotide includes a single nucleotide polymorphism (SNP) that is present in the diseased allele but not in the counterpart wild-type allele. Thus, e.g., in some cases, the disease-associated mutation-containing allele comprises a SNP that is not present in the corresponding wild-type allele, and the method comprises contacting the target nucleic acid with: a) a CRISPR-Cas effector polypeptide or a fusion polypeptide comprising a CRISPR-Cas effector polypeptide; and b) a guide nucleic acid, wherein the guide nucleic acid comprises: i) a targeting region that comprises a nucleotide sequence that binds to a target sequence in the target nucleic acid, wherein the target sequence comprises the SNP; and ii) a protein-binding region that binds to the CRISPR-Cas effector polypeptide. Allele-specific modification is depicted schematically in FIG. 2. The use of SNPs to target the modification to a disease-associated mutation-containing allele is depicted in FIG. 9-FIG. 13. In some cases, the SNP is in a PAM sequence.

In some cases, the level of protein that is produced, that is encoded by the modified allele, is at least 5% (e.g., at least 5%, at least 10%, at least 20%, at least 25%, at least 30%, at least 40%, at least 50%, at least 60%, at least 70%, at least 80%, or more than 80%) lower than the level of protein that is produced, that is encoded by the wild-type (and unmodified) allele. For example, where the modified allele is a modified version of a disease-associated mutation containing allele, where the disease-associated mutation-containing allele encodes a toxic gain-of-function polypeptide, and where the corresponding (counterpart) wild-type allele encodes a normal, functional (non-disease-associated) version of the toxic gain-of-function polypeptide, the ratio of level of the non-disease-associated polypeptide to the level of the disease-associated polypeptide is greater than 1:1 (e.g., the ratio is from 1.5:1 to 2:1, from 2:1 to 5:1, from 5:1 to 10:1, or greater than 10:1).

SNPs that distinguish between a disease-associated mutation-containing allele and a counterpart wild-type allele (that does not contain the disease-associated mutation) are known in the art for various disease alleles. See, e.g., Kay et al. (2019) Am. J. Human Genetics 105:1112; and Kay et al. (2015) Mol. Ther. 23:1759; these references provide allele-specific SNPs that are found in the HTT gene that encodes huntingtin. See also, e.g., Varela et al. (2016) Eur. J. Hum. Genet. 24:271, and associated supplemental online files; this paper identifies SNPs associated with many repeat expansion diseases (amyotrophic lateral sclerosis and frontotemporal dementia, dentatorubral-pallidoluysian atrophy, myotonic dystrophy 1, myotonic dystrophy 2, Huntington's disease and several spinocerebellar ataxias. See also Prudencio et al. (2020) Sci. Transl. Med. 12:eabb7086; this paper describes an allele-specific SNP in spinocerebellar ataxin type 3. Non-limiting examples of SNPs that distinguish between an mHtt allele and a counterpart wild-type Htt allele can also be found in FIG. 9.

In some cases, a target nucleic acid is a nucleic acid that encodes a toxic gain-of-function polypeptide. In some cases, the target nucleic acid comprises a trinucleotide, a tetranucleotide, or a hexanucleotide repeat expansion, where the target nucleic acid encodes a toxic gain-of-function polypeptide. Examples include, e.g., a CAG trinucleotide repeat expansion (where such repeat expansions give rise to Huntington's disease, spinal and bulbar muscular atrophy, dentatorubral-pallidoluysian atrophy, and several spinocerebellar ataxias); CTG trinucleotide repeat expansions (where such repeat expansions give rise to myotonic dystrophy 1, spinocerebellar ataxia type 8, Fuchs corneal dystrophy, and Huntington's disease-like 2); GAA trinucleotide repeats (where such trinucleotide repeats give rise to Friedreich's ataxia); CGG trinucleotide expansion (where such repeat expansions in the give rise to Fragile X syndrome and Fragile X tremor ataxia syndrome, or oculopharyngodistal myopathy types 1-3); GGGGCC hexanucleotide repeat expansions (where such hexanucleotide repeat expansions give rise to C9orf72 frontotemporal dementia and amyotrophic lateral sclerosis); GGCCTG hexanucleotide repeat expansions (where such hexanucleotide repeat expansions give rise to spinocerebellar ataxia type 36); and the like. See, e.g., Paulson (2018) Handbook of Clinical Neurology 147:105.

### Homology-Directed Repair

In some cases, modification of a target nucleic acid to generate a modified target nucleic acid, where a spliced mRNA product of the modified target nucleic acid comprises a premature stop codon, is carried out using homology-directed repair (HDR). For example, in some cases, a target nucleic acid is contacted with: i) a CRISPR-Cas effector polypeptide; ii) a guide RNA; and iii) a donor nucleic acid, where the donor nucleic acid comprises a nucleotide sequence containing the desired sequence modification. Donor nucleic acids are discussed herein.

### Base Editing

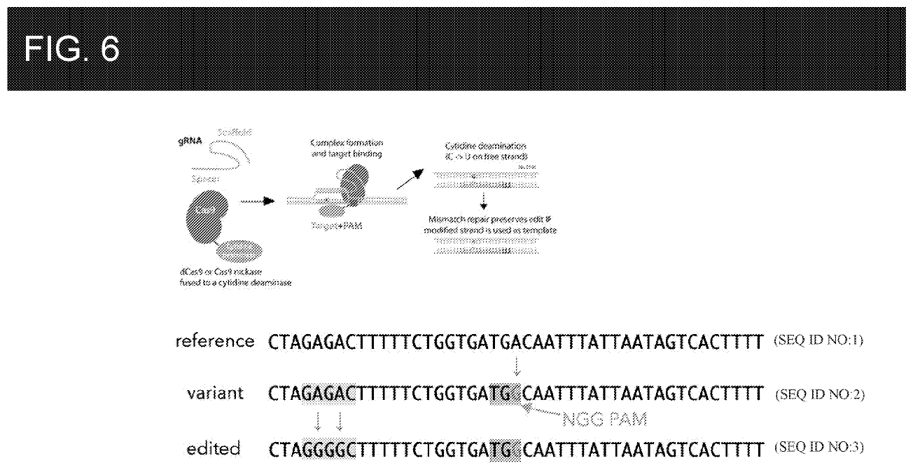

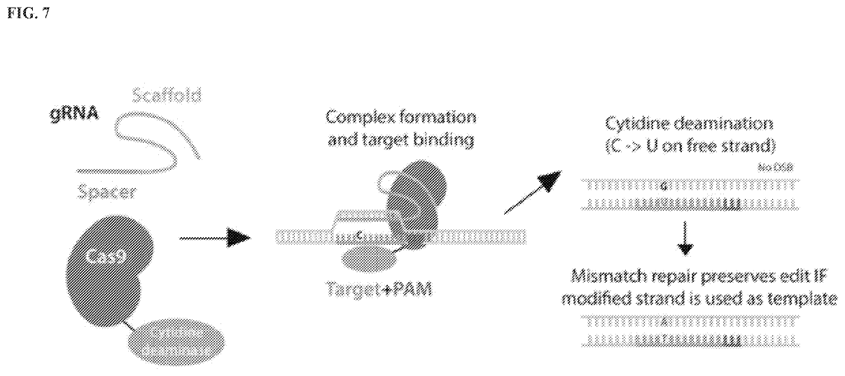

In some cases, modification of a target nucleic acid to generate a modified target nucleic acid, where a spliced mRNA product of the modified target nucleic acid comprises a premature stop codon, is carried out using base editing. Schematic depictions of gene editing using base editing are provided in FIG. 6 and FIG. 7. For example, in some cases, a target nucleic acid is contacted with: a) a CRISPR-Cas effector fusion polypeptide comprising: i) a CRISPR-Cas effector polypeptide (e.g., a CRISPR-Cas effector polypeptide that is a catalytically inactive CRISPR-Cas effector polypeptide (binds to, but does not cleave, a target nucleic acid) or a CRISPR-Cas effector polypeptide that is a nickase (binds to and cleaves only one strand of a target nucleic acid), where such a CRISPR-Cas effector fusion polypeptide is referred to as a “base editor”; and ii) one or more heterologous fusion partners, where at least one of the one or more heterologous fusion partners is a cytidine deaminase or an adenosine deaminase); and b) a guide nucleic acid. In some cases, the base editor effects a C G to T A base pair modification. In some cases, the base editor effects an A T to G C base pair modification.

### Prime Editing

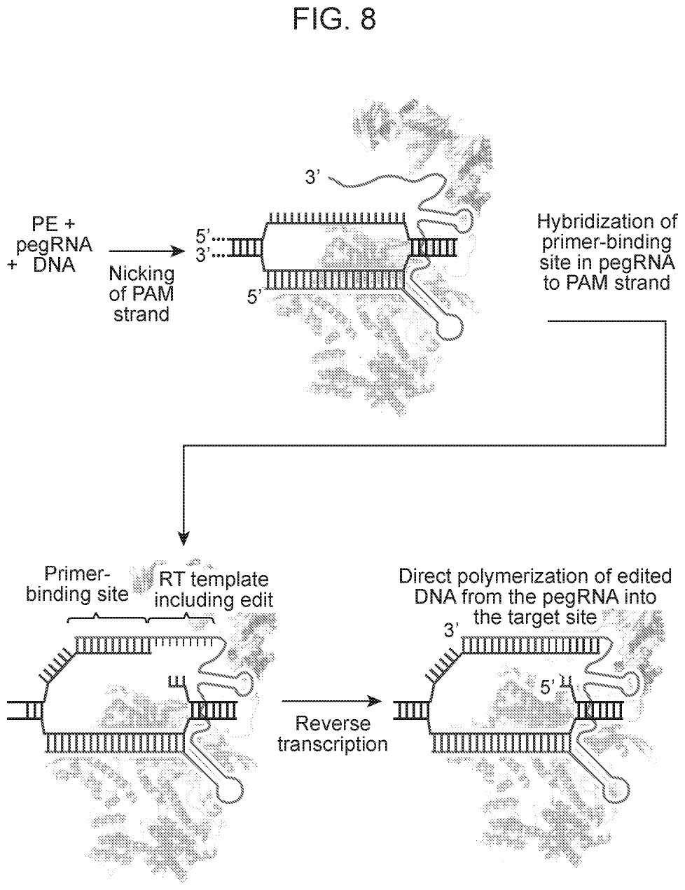

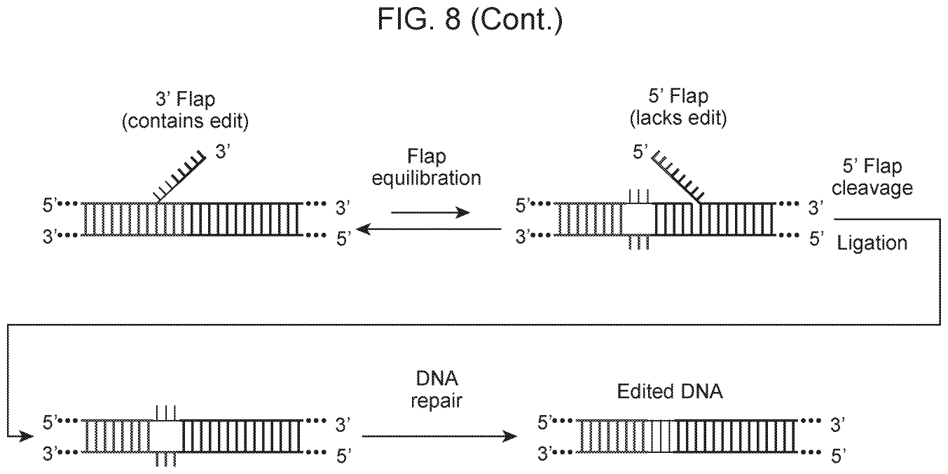

In some cases, modification of a target nucleic acid to generate a modified target nucleic acid, where a spliced mRNA product of the modified target nucleic acid comprises a premature stop codon, is carried out using prime editing. A schematic depiction of prime editing is provided in FIG. 8. Prime editing uses a catalytically-impaired CRISPR-Cas effector polypeptide that is fused to a reverse transcriptase polypeptide and programmed with a prime-editing guide RNA (pegRNA). The pegRNA includes a targeting region (comprising a nucleotide sequence that binds to a target nucleotide sequence in the target nucleic acid), a protein-binding region (comprising a nucleotide sequence that binds to the CRISPR-Cas effector polypeptide, and encodes the desired edit (e.g., a nucleotide sequence that gives rise to a poison exon). The catalytically-impaired CRISPR-Cas effector endonuclease is a nickase (cleaves only one strand of the target nucleic acid). During gene editing, the CRISPR-Cas effector polypeptide/reverse transcriptase fusion polypeptide is guided to the DNA target site by the pegRNA. The CRISPR-Cas effector polypeptide portion of the fusion protein nicks one strand of the target nucleic acid. The reverse transcriptase polypeptide then uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Afterward, the prime editor (PE) guides resolution of the heteroduplex to favor copying the edit onto the unedited strand, completing the process

Thus, in some cases, the genetic modification to generate a poison exon comprises use of a prime editor and a guide RNA. For example, in some cases, the target nucleic acid is contacted with: a) a CRISPR-Cas effector fusion polypeptide comprising: i) a CRISPR-Cas effector polypeptide, where the CRISPR-Cas effector polypeptide is a nickase; and ii) a reverse transcriptase polypeptide; b) prime-editing guide RNA (pegRNA), where the pegRNA comprises: i) a targeting region (i.e., a region comprising a nucleotide sequence that binds to a target nucleotide sequence in a target nucleic acid); ii) a protein-binding region comprising a nucleotide sequence that binds to the CRISPR-Cas effector polypeptide; iii) a primer binding nucleotide sequence; and iv) a poison exon nucleotide sequence (e.g., a complement of a poison exon). The CRISPR-Cas effector polypeptide present in the CRISPR-Cas effector fusion polypeptide nicks a strand of the target nucleic acid and the reverse transcriptase polypeptide present in the CRISPR-Cas effector fusion polypeptide incorporates the poison exon nucleotide sequence into the nicked site, thereby incorporating the poison exon nucleotide sequence into the target nucleic acid, thereby generating a modified target nucleic acid. In some cases, the method comprises introducing an expressible nucleic acid construct into a cell comprising the target nucleic acid, where the expressible nucleic acid construct comprises a nucleotide sequence encoding the CRISPR-Cas effector fusion polypeptide.

### CRISPR-Cas Effector Polypeptides

Suitable CRISPR-Cas effector polypeptides include Type II CRISPR-Cas effector polypeptides, Type III CRISPR Cas effector polypeptides, Type V CRISPR Cas effector polypeptides, and Type VI CRISPR-Cas effector polypeptides. Suitable CRISPR-Cas effector polypeptides include fusion polypeptides that comprise: i) a CRISPR-Cas effector polypeptide; and ii) one or more heterologous polypeptides.

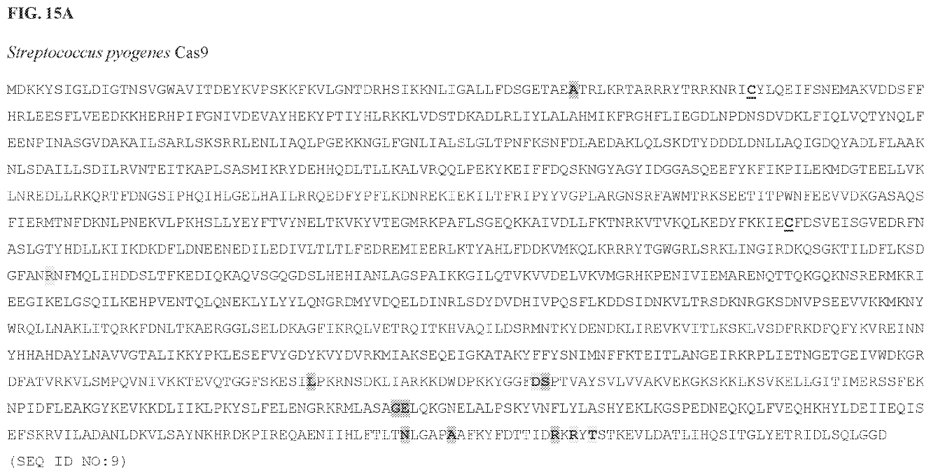

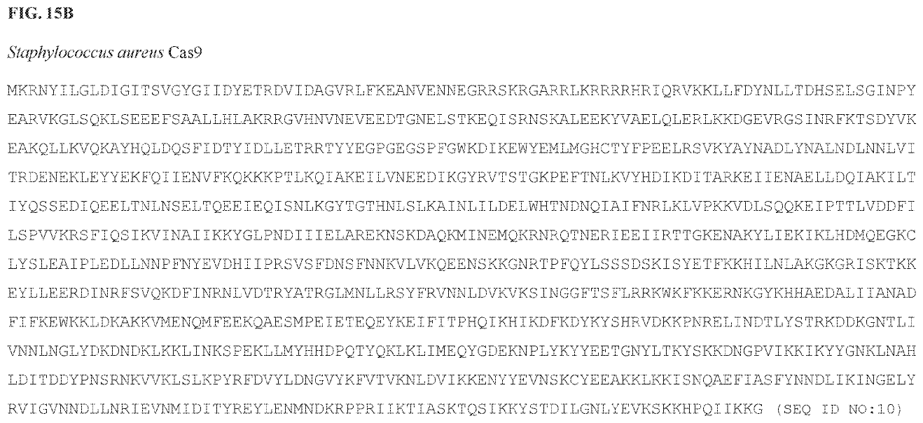

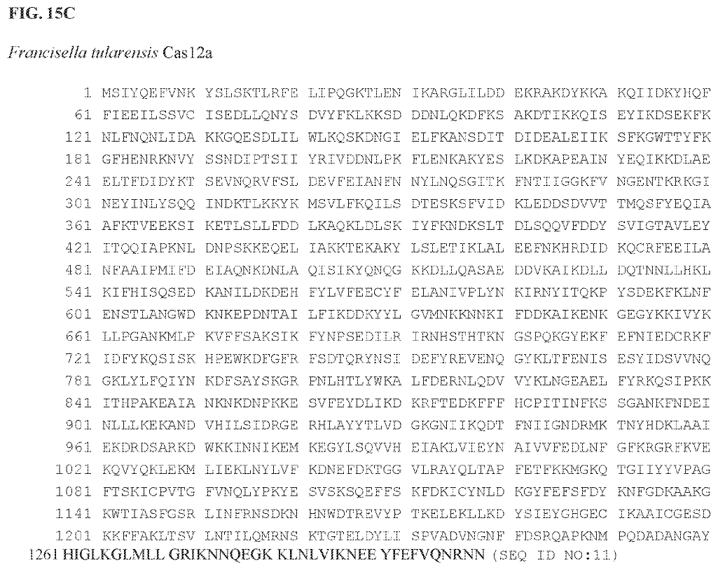

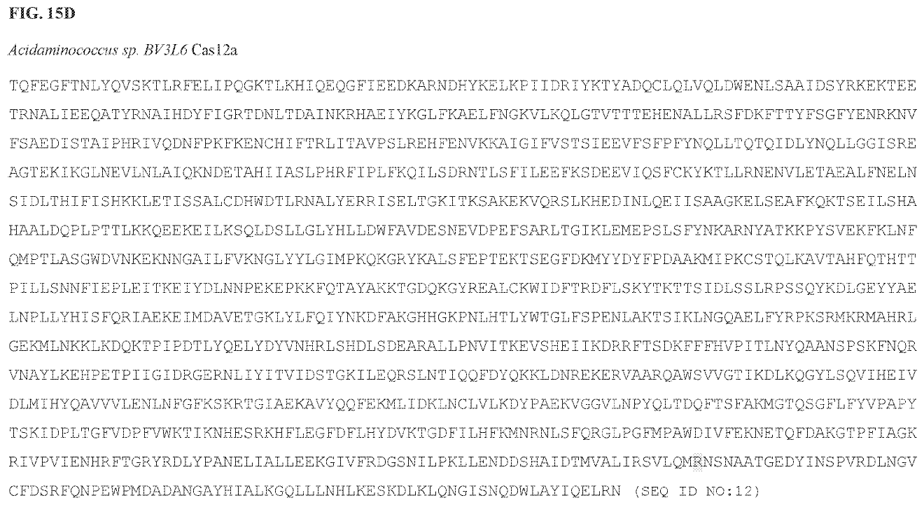

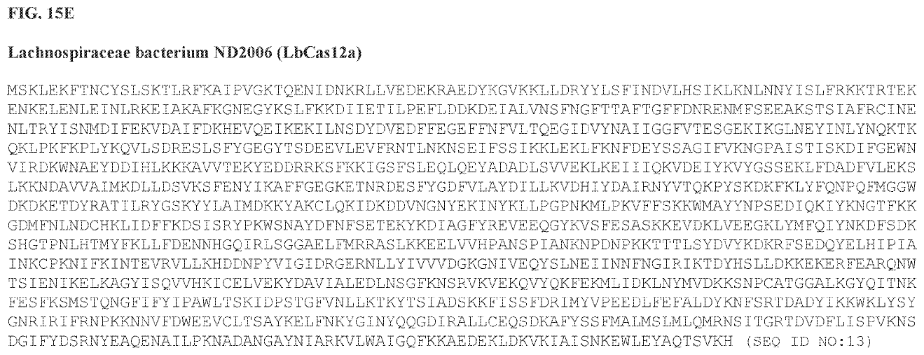

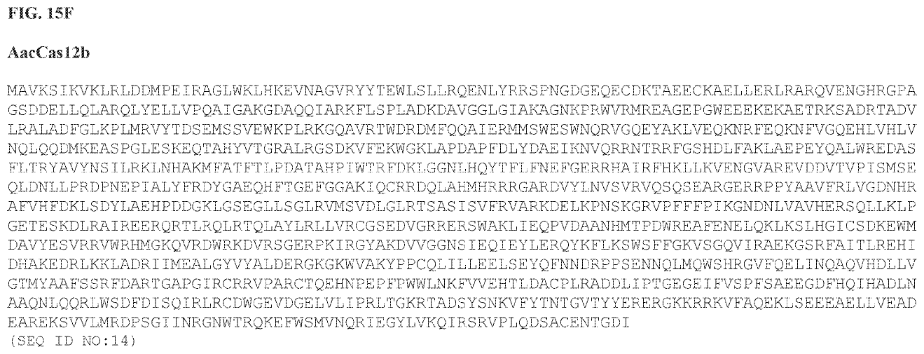

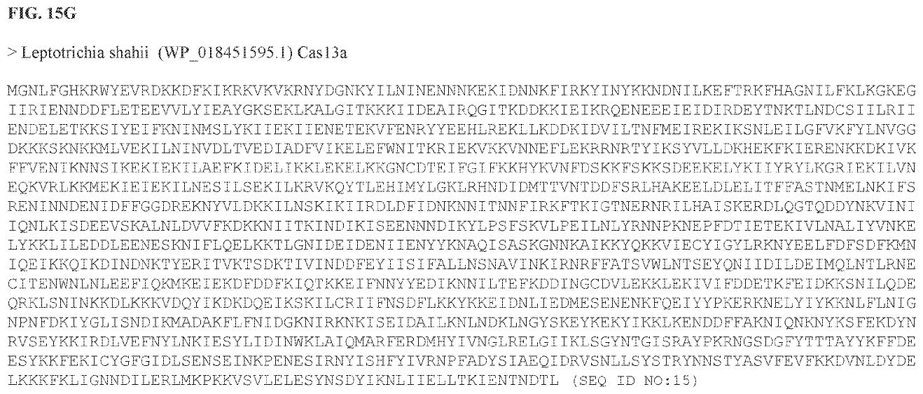

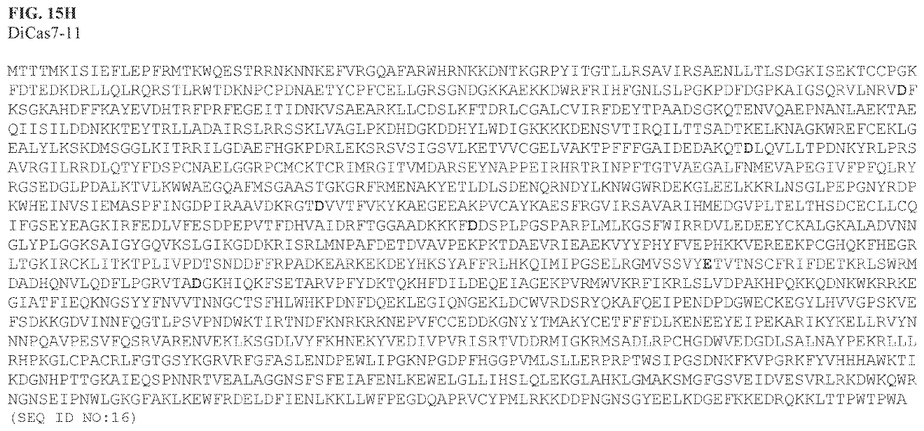

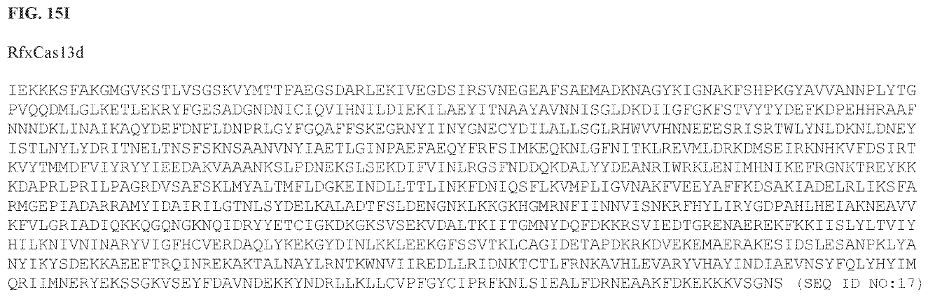

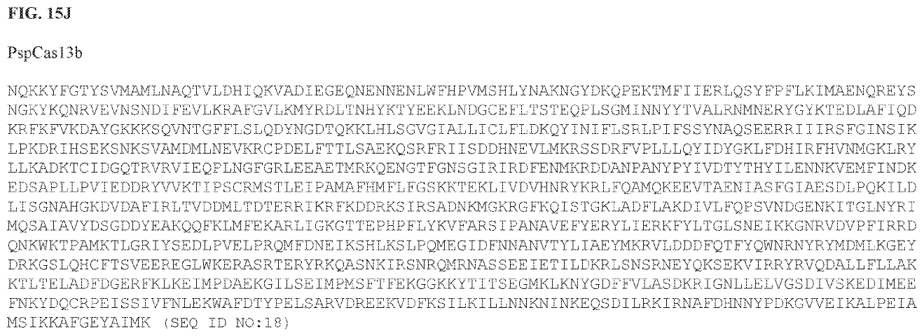

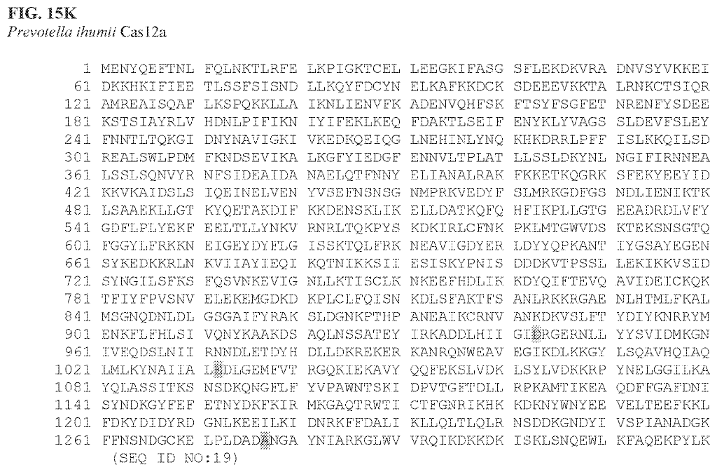

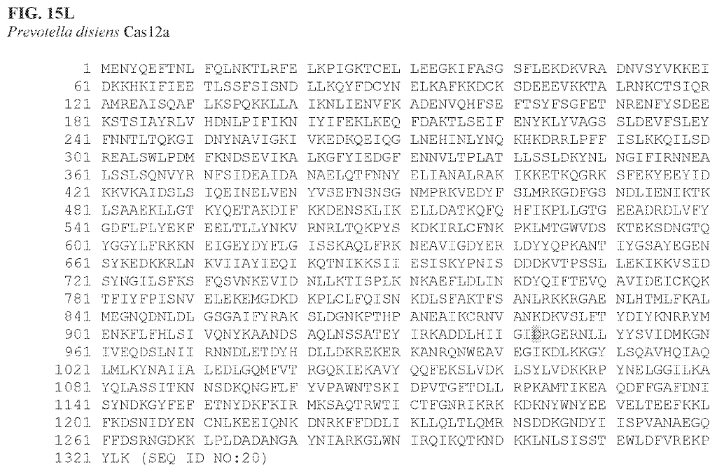

In some cases, the CRISPR-Cas effector polypeptide is a type II CRISPR-Cas effector polypeptide. In some cases, the type II CRISPR-Cas effector polypeptide is a Cas9 polypeptide, e.g., Staphylococcus aureus Cas9, Streptococcus pyogenes Cas9 (SpCas9), etc. In some cases, the CRISPR-Cas effector polypeptide is a variant of a wild-type SpCas9 and comprises one or more of the following substitutions: A61R, L1111R, A1322R, D1135L, S1136W, G1218K, E1219Q, N1317R, R1333P, R1335A, and T1337R. In some cases, the CRISPR-Cas effector polypeptide is an SpG polypeptide or a SpRY polypeptide; see, e.g., Walton et al. (2020) Science 368:290, and WO 2019/051097. For example, a suitable CRISPR-Cas effector polypeptide is an SpCas9 polypeptide includes D1135V, Ri135Q, and Ti137R substitutions, relative to wild-type SpCas9. As another example, a suitable CRISPR-Cas effector polypeptide is an SpCas9 polypeptide includes D1135V, R1335Q, T1337R, and G1218R substitutions, relative to wild-type SpCas9. As another example, a suitable CRISPR-Cas effector polypeptide is an SpCas9 polypeptide includes D1135L, S1136W, G1218K, E1219Q, R1335A, and T1337R substitutions, relative to wild-type SpCas9. As another example, a suitable CRISPR-Cas effector polypeptide is an SpCas9 polypeptide includes L1111R, A1322R, D1135L, S1136W, G1218K, E1219Q, R1335A, and T1337R substitutions, relative to wild-type SpCas9. As another example, a suitable CRISPR-Cas effector polypeptide is an SpCas9 polypeptide includes A61R, L1111R, A1322R, D1135L, S1136W, G1218K, E1219Q, N1317R, R1333P, R1335A, and T1337R substitutions, relative to wild-type SpCas9. The amino acid sequence of a wild-type SpCas9 polypeptide is provided in FIG. 15A.

In some cases, the CRISPR-Cas effector polypeptide is a type V CRISPR-Cas effector polypeptide, e.g., a Cas12a, a Cas12b, a Cas12c, a Cas12d, or a Cas12e polypeptide. In some cases, the CRISPR-Cas effector polypeptide is a type VI CRISPR-Cas effector polypeptide, e.g., a Cas13a polypeptide, a Cas13b polypeptide, a Cas13c polypeptide, or a Cas13d polypeptide. In some cases, the CRISPR-Cas effector polypeptide is a Cas14 polypeptide. In some cases, the CRISPR-Cas effector polypeptide is a Cas14a polypeptide, a Cas14b polypeptide, or a Cas14c polypeptide. For example, a suitable CRISPR-Cas effector polypeptide comprises an amino acid sequence having at least 50%, at least 60%, at least 70%, at least 80%, at least 85%, at least 90%, at least 95%, at least 98%, or at least 99%, amino acid sequence identity to the amino acid sequence depicted in any one of FIG. 15A-15L.

In some cases, the CRISPR-Cas effector polypeptide is a CRISPRi polypeptide; see, e.g., Qi et al. (2013) Cell 152:1173; and Jensen et al. (2021) Genome Research doi:10.1101/gr.275607.121. In some cases, the CRISPR-Cas effector polypeptide is a CRISPRa polypeptide; see, e.g., Jensen et al. (2021) Genome Research doi:10.1101/gr.275607.121; and Breinig et al. (2019) Nature Methods 16:51. In some cases, the CRISPR-Cas effector polypeptide is a CRISPRoff polypeptide. See, e.g., Nuñez et al. (2021) Cell 184:2503. In some cases, the CRISPR-Cas effector polypeptide is a nickase. In some cases, the CRISPR-Cas effector polypeptide exhibits reduced catalytic activity compared to a wild-type CRISPR-Cas effector polypeptide.

### CRISPR-Cas Effector Polypeptide Variants—Dead CRISPR-Cas Polypeptides and Nickases

In some cases, a CRISPR-Cas effector polypeptide suitable for use in a method, a system, or a composition of the present disclosure is a catalytically inactive CRISPR-Cas effector polypeptide, e.g., the CRISPR-Cas effector polypeptide, when complexed with a guide RNA, binds to a target nucleic acid but does not substantially cleave the target nucleic acid. In some cases, a CRISPR-Cas effector polypeptide suitable for use in a method, a system, or a composition of the present disclosure is a nickase CRISPR-Cas effector polypeptide, i.e., a CRISPR-Cas effector polypeptide that, when complexed with a guide RNA, binds to a target nucleic acid and cleaves only one strand of the target nucleic acid. Catalytically inactive CRISPR-Cas effector polypeptides are also known as “dead” CRISPR-Cas effector polypeptides. Catalytically inactive CRISPR-Cas effector polypeptides and nickase CRISPR-Cas effector polypeptides are known in the art. See, e.g., Brezgin et al. (2019) Int. J. Mol. Sci. 20:6041. For example, a Streptococcus pyogenes Cas9 polypeptide comprising amino acid substitutions in the RuvC1 and/or the HNH domains can be catalytically inactive; e.g., where the Cas9 polypeptide comprises a D10A and an H84A substitution. Where a CRISPR-Cas polypeptide comprises a HEPN domain, mutations in the HEPN domain can give rise to catalytically inactive CRISPR-Cas effector polypeptides. As another example, mutations in the RuvC domain of Prevotella ihumii Cas12a (“PiCas12a”) and Prevotella disiens Cas12a (“PdCas12a”) (e.g., where the mutations are D946A (D943 of the amino acid sequence depicted in FIG. 15K), E1035A (E1032 of the amino acid sequence depicted in FIG. 15K), and D1279A for PiCas12a (D1277 of the amino acid sequence depicted in FIG. 15K); and D943A for PdCas12a (see FIG. 15L) give rise to a catalytically inactive Cas12a; see, e.g., Jacobsen et al. (2020) Nucl. Acids Res. 48:5624. As another example, a variant Cas7-11 polypeptide comprises a substitution of one or more of D177, D429, D654, D758, E959, and D998 (where the amino acid numbering is as set forth in FIG. 15H). D177, D429, D654, D758, E959, and D998 are in bold in FIG. 15H.

### CRISPR-Cas Effector Fusion Polypeptides

As noted above, in some cases, the CRISPR-Cas effector polypeptide for use in a method, system, or composition of the present disclosure is a fusion polypeptide (“a CRISPR-Cas effector fusion polypeptide”) that comprises: i) a CRISPR-Cas effector polypeptide; and ii) one or more heterologous polypeptides. The term “heterologous polypeptide” is used interchangeably herein with “fusion partner.”

In some cases, the fusion partner (heterologous polypeptide) is a reverse transcriptase. In some cases, the fusion partner (heterologous polypeptide) is a deaminase. In some cases, the fusion partner is a nuclear localization signal (NLS).

### Reverse Transcriptases

In some cases, a CRISPR-Cas effector fusion polypeptide comprises: i) a CRISPR-Cas effector polypeptide; and ii) one or more heterologous polypeptides (one or more “fusion partners”), where at least one of the one or more heterologous polypeptides is a reverse transcriptase. Such a CRISPR-Cas effector fusion polypeptide optionally also includes one or more NLSs. In some cases, the CRISPR-Cas effector polypeptide is catalytically inactive. In some cases, the CRISPR-Cas effector polypeptide is a nickase (e.g., cleaves only one strand of a double-stranded target DNA). Reverse transcriptases are known in the art; see, e.g., Coté and Roth (2008) Virus Res. 134:186. Suitable reverse transcriptases include, e.g., a murine leukemia virus reverse transcriptase; a Rous sarcoma virus reverse transcriptase; a human immunodeficiency virus type I reverse transcriptase; a Moloney murine leukemia virus reverse transcriptase; a transcription xenopolymerase (RTX); avian myeloblastosis virus reverse transcriptase (AMV-RT); a Eubacterium rectale maturase reverse transcriptase (Marathon®; and the like. The reverse transcriptase fusion partner can include one or more mutations. For example, in some cases, the reverse transcriptase is a M-MLV reverse transcriptase polypeptide that comprises one or more mutations selected from the group consisting of D200N, T306K, W313F, T330P and L603W.

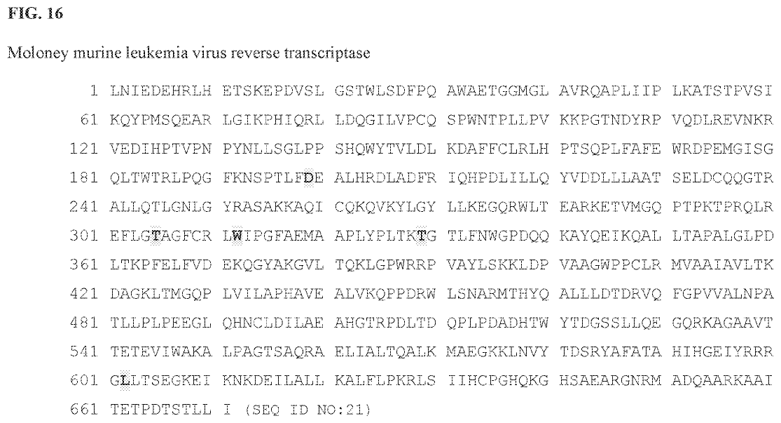

A CRISPR-Cas effector fusion polypeptide comprising: i) a CRISPR-Cas effector polypeptide, where the CRISPR-Cas effector polypeptide is a nickase; and ii) a reverse transcriptase, is referred as a “prime editor” (“PE”). In some cases, the CRISPR-Cas effector polypeptide is a Cas9 polypeptide comprising an H840A substitution. In some cases, the CRISPR-Cas effector polypeptide is a Cas12a/b nickase. In some cases, the reverse transcriptase is a pentamutant of M-MLV RT (e.g., comprising the following substitutions: D200N/L603W/T330P/T306K/W313F) (where D200, L603, T330, T306, and W313 correspond to D199, L602, T329, T305, and W312 of the M-MLV RT amino acid sequence depicted in FIG. 16).

In some cases, a suitable reverse transcriptase comprises an amino acid sequence having at least 50%, at least 60%, at least 70%, at least 80%, at least 85%, at least 90%, at least 95%, at least 98%, at least 99%, or 100%, amino acid sequence identity to the reverse transcriptase amino acid sequence depicted in FIG. 16.

### Base Editors

In some cases, a CRISPR-Cas effector fusion polypeptide comprises: i) a CRISPR-Cas effector polypeptide; and ii) one or more heterologous polypeptides (one or more “fusion partners”), where at least one of the one or more heterologous polypeptides is a deaminase. Such a CRISPR-Cas effector fusion polypeptide optionally also includes one or more NLSs. In some cases, the CRISPR-Cas effector polypeptide is catalytically inactive. In some cases, the CRISPR-Cas effector polypeptide is a nickase (e.g., cleaves only one strand of a double-stranded target DNA). Suitable base editors include, e.g., an adenosine deaminase; a cytidine deaminase (e.g., an activation-induced cytidine deaminase (AID)); APOBEC3G; and the like); and the like. A suitable adenosine deaminase is any enzyme that is capable of deaminating adenosine in DNA. In some cases, the deaminase is a TadA deaminase.

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 23)
MSEVEFSHEYWMRHALTLAKRAWDEREVPVGAVLVHNNRVIGEGW
NRPIGRHDPTAHAEIMALRQGGLVMQNYRLIDATLYVTLEPCVMC
AGAMIHSRIGRVVFGARDAKTGAAGSLMDVLHHPGMNHRVEITEG
ILADECAALLSDFFRMRRQEIKAQKKAQSSTD

In some cases, a suitable adenosine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 24)
MRRAFITGVFFLSEVEFSHEYWMRHALTLAKRAWDEREVPVGAVL
VHNNRVIGEGWNRPIGRHDPTAHAEIMALRQGGLVMQNYRLIDAT
LYVTLEPCVMCAGAMIHSRIGRVVFGARDAKTGAAGSLMDVLHHP
GMNHRVEITEGILADECAALLSDFFRMRRQEIKAQKKAQSSTD.

In some cases, a suitable adenosine deaminase comprises an amino acid sequence having at least 80%, at least 85%, at least 90%, at least 95%, at least 98%, at least 99%, or 100%, amino acid sequence identity to the following Staphylococcus aureus TadA amino acid sequence:

(SEQ ID NO: 25)
MGSHMTNDIYFMTLAIEEAKKAAQLGEVPIGAIITKDDEVIARAH
NLRETLQQPTAHAEHIAIERAAKVLGSWRLEGCTLYVTLEPCVMC
AGTIVMSRIPRVVYGADDPKGGCSGSLMNLLQQSNFNHRAIVDKG
VLKEACSTLLTTFFKNLRANKKSTN:

In some cases, a suitable adenosine deaminase comprises an amino acid sequence having at least 80%, at least 85%, at least 90%, at least 95%, at least 98%, at least 99%, or 100%, amino acid sequence identity to the following Bacillus subtilis TadA amino acid sequence:

(SEQ ID NO: 26)
MTQDELYMKEAIKEAKKAEEKGEVPIGAVLVINGEIIARAHNLRE
TEQRSIAHAEMLVIDEACKALGTWRLEGATLYVTLEPCPMCAGAV
VLSRVEKVVFGAFDPKGGCSGTLMNLLQEERFNHQAEVVSGVLEE
ECGGMLSAFFRELRKKKKAARKNLSE

In some cases, a suitable adenosine deaminase comprises an amino acid sequence having at least 80%, at least 85%, at least 90%, at least 95%, at least 98%, at least 99%, or 100%, amino acid sequence identity to the following Salmonella typhimurium TadA:

(SEQ ID NO: 27)
MPPAFITGVTSLSDVELDHEYWMRHALTLAKRAWDEREVPVGAVL
VHNHRVIGEGWNRPIGRHDPTAHAEIMALRQGGLVLQNYRLLDTT
LYVTLEPCVMCAGAMVHSRIGRVVFGARDAKTGAAGSLIDVLHHP
GMNHRVEIIEGVLRDECATLLSDFFRMRRQEIKALKKADRAEGAG
PAV

In some cases, a suitable adenosine deaminase comprises an amino acid sequence having at least 80%, at least 85%, at least 90%, at least 95%, at least 98%, at least 99%, or 100%, amino acid sequence identity to the following Shewanella putrefaciens TadA amino acid sequence:

(SEQ ID NO: 28)
MDEYWMQVAMQMAEKAEAAGEVPVGAVLVKDGQQIATGYNLSISQ
HDPTAHAEILCLRSAGKKLENYRLLDATLYITLEPCAMCAGAMVH
SRIARVVYGARDEKTGAAGTVVNLLQHPAFNHQVEVTSGVLAEAC
SAQLSRFFKRRRDEKKALKLAQRAQQGIE

In some cases, a suitable adenosine deaminase comprises an amino acid sequence having at least 80%, at least 85%, at least 90%, at least 95%, at least 98%, at least 99%, or 100%, amino acid sequence identity to the following Haemophilus influenzae F3031 TadA amino acid sequence:

(SEQ ID NO: 29)
MDAAKVRSEFDEKMMRYALELADKAEALGEIPVGAVLVDDARNII
GEGWNLSIVQSDPTAHAEIIALRNGAKNIQNYRLLNSTLYVTLEP
CTMCAGAILHSRIKRLVFGASDYKTGAIGSRFHFEDDYKMNHTLE
ITSGVLAEECSQKLSTFFQKRREEKKIEKALLKSLSDK

In some cases, a suitable adenosine deaminase comprises an amino acid sequence having at least 80%, at least 85%, at least 90%, at least 95%, at least 98%, at least 99%, or 100%, amino acid sequence identity to the following Caulobacter crescentus TadA amino acid sequence:

(SEQ ID NO: 30)
MRTDESEDQDHRMMRLALDAARAAAEAGETPVGAVILDPSTGEVI
ATAGNGPIAAHDPTAHAEIAAMRAAAAKLGNYRLTDLTLVVTLEP
CAMCAGAISHARIGRVVFGADDPKGGAVVHGPKFFAQPTCHWRPE
VTGGVLADESADLLRGFFRARRKAKI

In some cases, a suitable adenosine deaminase comprises an amino acid sequence having at least 80%, at least 85%, at least 90%, at least 95%, at least 98%, at least 99%, or 100%, amino acid sequence identity to the following Geobacter sulfurreducens TadA amino acid sequence:

(SEQ ID NO: 31)
MSSLKKTPIRDDAYWMGKAIREAAKAAARDEVPIGAVIVRDGAVI
GRGHNLREGSNDPSAHAEMIAIRQAARRSANWRLTGATLYVTLEP
CLMCMGAIILARLERVVFGCYDPKGGAAGSLYDLSADPRLNHQVR
LSPGVCQEECGTMLSDFFRDLRRRKKAKATPALFIDERKVPPEP

Cytidine deaminases suitable for inclusion in a Cas12L fusion polypeptide include any enzyme that is capable of deaminating cytidine in DNA.

In some cases, the cytidine deaminase is a deaminase from the apolipoprotein B mRNA-editing complex (APOBEC) family of deaminases. In some cases, the APOBEC family deaminase is selected from the group consisting of APOBEC1 deaminase, APOBEC2 deaminase, APOBEC3A deaminase, APOBEC3B deaminase, APOBEC3C deaminase, APOBEC3D deaminase, APOBEC3F deaminase, APOBEC3G deaminase, and APOBEC3H deaminase. In some cases, the cytidine deaminase is an activation induced deaminase (AID).

In some cases, a suitable cytidine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 32)
MDSLLMNRRKFLYQFKNVRWAKGRRETYLCYVVKRRDSATSFSLD
FGYLRNKNGCHVELLFLRYISDWDLDPGRCYRVTWFTSWSPCYDC
ARHVADFLRGNPNLSLRIFTARLYFCEDRKAEPEGLRRLHRAGVQ
IAIMTFKDYFYCWNTFVENHERTFKAWEGLHENSVRLSRQLRRIL
LPLYEVDDLRDAFRTLGL

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKENHERTFK AWEGLHENSV RLSRQLRRIL LPLYEVDDLR DAFRTLGL (SEQ ID NO:33).

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKDYFYCWNT FVENHERTFK AWEGLHENSV RLSRQLRRIL LPLYEVDDLR DAFRTLGL (SEQ ID NO:32).

### NLSs and CPPs

In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some cases, a CRISPR-Cas effector fusion polypeptide does not include an NLS so that the protein is not targeted to the nucleus (which can be advantageous, e.g., when the target nucleic acid is an RNA that is present in the cytosol). In some cases, the heterologous polypeptide can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, a CRISPR-Cas effector protein (e.g., a wild type CRISPR-Cas effector protein, a variant CRISPR-Cas effector protein, a fusion CRISPR-Cas effector protein, a dCRISPR-Cas effector protein, a nickase CRISPR-Cas effector protein, and the like) includes (is fused to) a nuclear localization signal (NLS) (e.g., in some cases 2 or more, 3 or more, 4 or more, or 5 or more NLSs). Thus, in some cases, a CRISPR-Cas effector polypeptide includes one or more NLSs (e.g., 2 or more, 3 or more, 4 or more, or 5 or more NLSs). In some cases, one or more NLSs (2 or more, 3 or more, 4 or more, or 5 or more NLSs) are positioned at or near (e.g., within 50 amino acids of) the N-terminus and/or the C-terminus. In some cases, one or more NLSs (2 or more, 3 or more, 4 or more, or 5 or more NLSs) are positioned at or near (e.g., within 50 amino acids of) the N-terminus. In some cases, one or more NLSs (2 or more, 3 or more, 4 or more, or 5 or more NLSs) are positioned at or near (e.g., within 50 amino acids of) the C-terminus. In some cases, one or more NLSs (3 or more, 4 or more, or 5 or more NLSs) are positioned at or near (e.g., within 50 amino acids of) both the N-terminus and the C-terminus. In some cases, an NLS is positioned at the N-terminus and an NLS is positioned at the C-terminus.

In some cases, a CRISPR-Cas effector protein (e.g., a wild type CRISPR-Cas effector protein, a variant CRISPR-Cas effector protein, a fusion CRISPR-Cas effector protein, a dCRISPR-Cas effector protein, and the like) includes (is fused to) between 1 and 10 NLSs (e.g., 1-9, 1-8, 1-7, 1-6, 1-5, 2-10, 2-9, 2-8, 2-7, 2-6, or 2-5 NLSs). In some cases, a CRISPR-Cas effector protein (e.g., a wild type CRISPR-Cas effector protein, a variant CRISPR-Cas effector protein, a fusion CRISPR-Cas effector protein, a dCRISPR-Cas effector protein, and the like) includes (is fused to) between 2 and 5 NLSs (e.g., 2-4, or 2-3 NLSs).

Non-limiting examples of NLSs include an NLS sequence derived from: the NLS of the SV40 virus large T-antigen, having the amino acid sequence PKKKRKV (SEQ ID NO:34); the NLS from nucleoplasmin (e.g., the nucleoplasmin bipartite NLS with the sequence KRPAATKKAGQAKKKK (SEQ ID NO:35)); the c-myc NLS having the amino acid sequence PAAKRVKLD (SEQ ID NO:36) or RQRRNELKRSP (SEQ ID NO:37); the hRNPA1 M9 NLS having the sequence NQSSNFGPMKGGNFGGRSSGPYGGGGQYFAKPRNQGGY (SEQ ID NO:38); the sequence RMRIZFKNKGKDTAELRRRRVEVSVELRKAKKDEQILKRRNV (SEQ ID NO:39) of the IBB domain from importin-alpha; the sequences VSRKRPRP (SEQ ID NO:40) and PPKKARED (SEQ ID NO:41) of the myoma T protein; the sequence PQPKKKPL (SEQ ID NO:42) of human p53; the sequence SALIKKKKKMAP (SEQ ID NO:43) of mouse c-abl IV; the sequences DRLRR (SEQ ID NO:44) and PKQKKRK (SEQ ID NO:45) of the influenza virus NS1; the sequence RKLKKKIKKL (SEQ ID NO:46) of the Hepatitis virus delta antigen; the sequence REKKKFLKRR (SEQ ID NO:47) of the mouse Mxl protein; the sequence KRKGDEVDGVDEVAKKKSKK (SEQ ID NO:48) of the human poly(ADP-ribose) polymerase; and the sequence RKCLQAGMNLEARKTKK (SEQ ID NO:49) of the steroid hormone receptors (human) glucocorticoid. In general, NLS (or multiple NLSs) are of sufficient strength to drive accumulation of the CRISPR-Cas effector protein in a detectable amount in the nucleus of a eukaryotic cell. Detection of accumulation in the nucleus may be performed by any suitable technique. For example, a detectable marker may be fused to the CRISPR-Cas effector protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

In some cases, a CRISPR-Cas effector fusion polypeptide includes a “Protein Transduction Domain” or PTD (also known as a CPP—cell penetrating peptide), which refers to a polypeptide, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from extracellular space to intracellular space, or cytosol to within an organelle. In some embodiments, a PTD is covalently linked to the amino terminus a polypeptide (e.g., linked to a wild type CRISPR-Cas effector to generate a fusion protein, or linked to a variant CRISPR-Cas effector protein such as a dCRISPR-Cas effector, a nickase CRISPR-Cas effector, or a fusion CRISPR-Cas effector protein, to generate a fusion protein). In some embodiments, a PTD is covalently linked to the carboxyl terminus of a polypeptide (e.g., linked to a wild type CRISPR-Cas effector to generate a fusion protein, or linked to a variant CRISPR-Cas effector protein such as a dCRISPR-Cas effector protein, a nickase CRISPR-Cas effector, or a fusion CRISPR-Cas effector protein to generate a fusion protein). In some cases, the PTD is inserted internally in the CRISPR-Cas effector fusion polypeptide (i.e., is not at the N- or C-terminus of the CRISPR-Cas effector fusion polypeptide) at a suitable insertion site. In some cases, a subject CRISPR-Cas effector fusion polypeptide includes (is conjugated to, is fused to) one or more PTDs (e.g., two or more, three or more, four or more PTDs). In some cases, a PTD includes a nuclear localization signal (NLS) (e.g., in some cases 2 or more, 3 or more, 4 or more, or 5 or more NLSs). Thus, in some cases, a CRISPR-Cas effector fusion polypeptide includes one or more NLSs (e.g., 2 or more, 3 or more, 4 or more, or 5 or more NLSs). In some embodiments, a PTD is covalently linked to a nucleic acid (e.g., a CRISPR-Cas effector guide nucleic acid, a polynucleotide encoding a CRISPR-Cas effector guide nucleic acid, a polynucleotide encoding a CRISPR-Cas effector fusion polypeptide, a donor polynucleotide, etc.). Examples of PTDs include but are not limited to a minimal undecapeptide protein transduction domain (corresponding to residues 47-57 of HIV-1 TAT comprising YGRKKRRQRRR; SEQ ID NO:50); a polyarginine sequence comprising a number of arginines sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:51); Transportan GWTLNSAGYLLGKINLKALAALAKKIL (SEQ ID NO:52); KALAWEAKLAKALAKALAKHLAKALAKALKCEA (SEQ ID NO:53); and RQIKIWFQNRRMKWKK (SEQ ID NO:54). Exemplary PTDs include but are not limited to, YGRKKRRQRRR (SEQ ID NO:50), RKKRRQRRR (SEQ ID NO:55); an arginine homopolymer of from 3 arginine residues to 50 arginine residues; Exemplary PTD domain amino acid sequences include, but are not limited to, any of the following: YGRKKRRQRRR (SEQ ID NO:50); RKKRRQRR (SEQ ID NO:56); YARAAARQARA (SEQ ID NO:57); THRLPRRRRRR (SEQ ID NO:58); and GGRRARRRRRR (SEQ ID NO:59). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

### Linkers (e.g., for Fusion Partners)

In some embodiments, a CRISPR-Cas effector protein can be fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G)ₙ, glycine-serine polymers (including, for example, (GS)ₙ (SEQ ID NO:60), (GSGGS)ₙ (SEQ ID NO:61), (GGSGGS)ₙ (SEQ ID NO:62), and (GGGGS)ₙ (SEQ ID NO:63), where n is an integer of at least one, e.g., where n is 1, 2, 3, 4, 5, 6, 7, 8, 9, or 10), glycine-alanine polymers, alanine-serine polymers. Exemplary linkers can comprise amino acid sequences including, but not limited to, GGSG (SEQ ID NO:64), GGSGG (SEQ ID NO:65), GSGSG (SEQ ID NO:66), GSGGG (SEQ ID NO:67), GGGSG (SEQ ID NO:68), GSSSG (SEQ ID NO:69), and the like. The ordinarily skilled artisan will recognize that design of a peptide conjugated to any desired element can include linkers that are all or partially flexible, such that the linker can include a flexible linker as well as one or more portions that confer less flexible structure.

### CRISPR-Cas Guide Nucleic Acids

As noted above, a guide nucleic acid comprises: i) a binding region that binds to a CRISPR-Cas effector polypeptide; and ii) a targeting region that comprises a nucleotide sequence that is complementary to a target sequence of a target nucleic acid. In some cases, the binding region is heterologous to the targeting region. In some cases, the nucleotide sequence that is complementary to a target sequence of a target nucleic acid is 15 nucleotides to 18 nucleotides long. In some cases, the nucleotide sequence that is complementary to a target sequence of a target nucleic acid is 18 nucleotides to 25 nucleotides long. In some cases, the targeting region has a length of from 15 nucleotides to 30 nucleotides (e.g., from 15 to 20, from 17 to 25, from 17 to 22, from 18 to 22, from 18 to 20, from 20 to 25, or from 25 to 30 nucleotides). In some cases, e.g., where the guide nucleic acid is to be used with a prime editor, the guide nucleic acid comprises: i) a binding region that binds to a CRISPR-Cas effector polypeptide; ii) a targeting region that comprises a nucleotide sequence that is complementary to a target sequence of a target nucleic acid; iii) a primer binding sequence; and iv) a nucleotide sequence that is desired to be incorporated into a target nucleic acid (e.g., a complement of the nucleotide sequence that is desired to be incorporated into a target nucleic acid).

In some cases, a CRISPR-Cas effector guide RNA has one or more modifications, e.g., one or more of a base modification, a backbone modification, and a sugar modification.

Suitable modified backbones containing a phosphorus atom therein include, for example, phosphorothioates, chiral phosphorothioates, phosphorodithioates, phosphotriesters, aminoalkylphosphotriesters, methyl and other alkyl phosphonates including 3-alkylene phosphonates, 5′-alkylene phosphonates and chiral phosphonates, phosphinates, phosphoramidates including 3-amino phosphoramidate and aminoalkylphosphoramidates, phosphorodiamidates, thionophosphoramidates, thionoalkylphosphonates, thionoalkylphosphotriesters, selenophosphates and boranophosphates having normal 3-5′ linkages, 2′-5′ linked analogs of these, and those having inverted polarity wherein one or more internucleotide linkages is a 3′ to 3′, 5′ to 5′ or 2′ to 2′ linkage. Suitable nucleic acids having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

Suitable polynucleotides comprise a sugar substituent group selected from: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂)ₙOCH₃, O(CH₂)NH₂, O(CH₂)CH₃, O(CH₂)ₙONH₂, and O(CH₂)ₙON((CH₂)CH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504, the disclosure of which is incorporated herein by reference in its entirety) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

A subject nucleic acid may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), N1-methylpseudouridine, 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-(b) (1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one).

### Donor Nucleic Acids

As noted above, in some cases, a method of the present disclosure involves use of a donor nucleic acid. In some cases, a system of the present disclosure comprises a donor nucleic acid. In some cases, a kit of the present disclosure comprises a donor nucleic acid.

Guided by a CRISPR-Cas effector guide RNA, a CRISPR-Cas effector protein in some cases generates site-specific double strand breaks (DSBs) or single strand breaks (SSBs) (e.g., when the CRISPR-Cas effector protein is a nickase variant) within double-stranded DNA (dsDNA) target nucleic acids. Such breaks can be repaired by non-homologous end joining (NHEJ) or homology-directed recombination (HDR).

In some cases, contacting a target nucleic acid (with a CRISPR-Cas effector protein and a CRISPR-Cas effector guide RNA) occurs under conditions that are permissive for nonhomologous end joining or homology-directed repair. Thus, in some cases, a subject method includes contacting a target DNA with a donor polynucleotide (e.g., by introducing the donor polynucleotide into a cell), wherein the donor polynucleotide, a portion of the donor polynucleotide, a copy of the donor polynucleotide, or a portion of a copy of the donor polynucleotide integrates into the target DNA. In some cases, the method does not comprise contacting a cell with a donor polynucleotide.

In some cases, CRISPR-Cas effector guide RNA (or DNA encoding same) and a CRISPR-Cas effector protein (or a nucleic acid encoding same, such as an RNA or a DNA, e.g., one or more expression vectors) are co-administered (e.g., contacted with a target nucleic acid, administered to cells, etc.) with a donor polynucleotide sequence that includes at least a segment with homology to the target DNA sequence, the subject methods may be used to modify the target nucleic acid, as described above, such that a splice acceptor or splice donor site is generated at a location where such splice acceptor or splice donor does not naturally occur in the target nucleic acid (such that a spliced mRNA product of the modified target nucleic acid includes a premature stop codon), or the subject methods may be used to introduce into a target nucleic acid a stop codon, or the subject methods may be used to introduce into a target nucleic acid a micro poison exon.

By a “donor sequence,” or “donor polynucleotide,” or “donor nucleic acid,” or “donor template” it is meant a nucleic acid to be inserted at the site cleaved by the CRISPR-Cas effector protein (e.g., after dsDNA cleavage, after nicking a target DNA, after dual nicking a target DNA, and the like). The donor polynucleotide can contain sufficient homology to a genomic sequence at the target site, e.g. 70%, 80%, 85%, 90%, 95%, or 100% homology with the nucleotide sequences flanking the target site, e.g. within about 50 bases or less of the target site, e.g. within about 30 bases, within about 15 bases, within about 10 bases, within about 5 bases, or immediately flanking the target site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) can support homology-directed repair. Donor polynucleotides can be of any length, e.g. 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair (e.g., for gene correction, e.g., to convert a disease-causing base pair to a non-disease-causing base pair). In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

In some cases, the donor nucleic acid is provided as single-stranded DNA. In some cases, the donor nucleic acid is provided as double-stranded DNA. A donor nucleic acid may be introduced into a cell in linear or circular form. If introduced in linear form, the ends of the donor nucleic acid may be protected (e.g., from exonucleolytic degradation) by any convenient method and such methods are known to those of skill in the art. For example, one or more dideoxynucleotide residues can be added to the 3′ terminus of a linear molecule and/or self-complementary oligonucleotides can be ligated to one or both ends. See, for example, Chang et al. (1987) Proc. Natl. Acad Sci USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. As an alternative to protecting the termini of a linear donor sequence, additional lengths of sequence may be included outside of the regions of homology that can be degraded without impacting recombination. A donor nucleic acid can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described elsewhere herein for nucleic acids encoding a CRISPR-Cas effector guide RNA and/or a CRISPR-Cas effector fusion polypeptide and/or donor polynucleotide.

### Systems

The present disclosure provides a system, which system may be used for carrying out a method of the present disclosure.

In some cases, a system of the present disclosure comprises: a) a CRISPR-Cas effector polypeptide, or a nucleic acid comprising a nucleotide sequence encoding a CRISPR-Cas effector polypeptide; b) a guide RNA comprising i) a targeting region that comprises a nucleotide sequence that binds to a target sequence in the target nucleic acid; and ii) a protein-binding region that binds to the CRISPR-Cas effector polypeptide; and c) a donor nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′: i) a nucleotide sequence of from about 5 nucleotides to about 15 nucleotides in length and having a first consensus splice motif; ii) a nucleotide sequence of from about 11 nucleotides to about 15 nucleotides in length and comprising a stop codon in each reading frame; and iii) a nucleotide sequence of from about 5 nucleotides to about 15 nucleotides in length and having a second consensus splice motif. A first consensus splice motif may also be referred to as an acceptor splice motif. A second consensus splice motif may also be referred to as a donor splice motif.

In some cases, a system of the present disclosure comprises: a) a nucleic acid (e.g., an expression construct) comprising a nucleotide sequence encoding a CRISPR-Cas effector polypeptide; b) a guide RNA comprising i) a targeting region that comprises a nucleotide sequence that binds to a target sequence in the target nucleic acid; and ii) a protein-binding region that binds to the CRISPR-Cas effector polypeptide; and c) a donor nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′: i) a nucleotide sequence of from about 5 nucleotides to about 15 nucleotides in length and having a first consensus splice motif; ii) a nucleotide sequence of from about 11 nucleotides to about 15 nucleotides in length and comprising a stop codon in each reading frame; and iii) a nucleotide sequence of from about 5 nucleotides to about 15 nucleotides in length and having a second consensus splice motif.

In some cases, a system of the present disclosure comprises: a) a CRISPR-Cas effector fusion polypeptide, wherein the CRISPR-Cas effector fusion polypeptide comprises: i) a CRISPR-Cas effector polypeptide; and ii) one or more heterologous fusion partners; b) a guide RNA comprising i) a targeting region that comprises a nucleotide sequence that binds to a target sequence in the target nucleic acid; ii) a protein-binding region that binds to the CRISPR-Cas effector polypeptide; and ii) a nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′: i) a nucleotide sequence of from about 5 nucleotides to about 15 nucleotides in length and having a first consensus splice motif; ii) a nucleotide sequence of from about 11 nucleotides to about 15 nucleotides in length and comprising a stop codon in each reading frame; and iii) a nucleotide sequence of from about 5 nucleotides to about 15 nucleotides in length and having a second consensus splice motif. In some cases, the one or more heterologous polypeptides comprises a reverse transcriptase and the guide RNA comprises a primer binding nucleotide sequence. In some cases, the CRISPR-Cas effector polypeptide is a nickase. In some cases, the one or more heterologous polypeptides comprises a cytidine deaminase. In some cases, the one or more heterologous polypeptides comprises an adenosine deaminase.

In some cases, a system of the present disclosure comprises: a) CRISPR- a nucleic acid (e.g., an expression construct) comprising a nucleotide sequence encoding a Cas effector fusion polypeptide, wherein the CRISPR-Cas effector fusion polypeptide comprises: i) a CRISPR-Cas effector polypeptide; and ii) one or more heterologous fusion partners; b) a guide RNA comprising i) a targeting region that comprises a nucleotide sequence that binds to a target sequence in the target nucleic acid; ii) a protein-binding region that binds to the CRISPR-Cas effector polypeptide; and ii) a nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′: i) a nucleotide sequence of from about 5 nucleotides to about 15 nucleotides in length and having a first consensus splice motif; ii) a nucleotide sequence of from about 11 nucleotides to about 15 nucleotides in length and comprising a stop codon in each reading frame; and iii) a nucleotide sequence of from about 5 nucleotides to about 15 nucleotides in length and having a second consensus splice motif. In some cases, the one or more heterologous polypeptides comprises a reverse transcriptase and the guide RNA comprises a primer binding nucleotide sequence. In some cases, the CRISPR-Cas effector polypeptide is a nickase. In some cases, the one or more heterologous polypeptides comprises a cytidine deaminase. In some cases, the one or more heterologous polypeptides comprises an adenosine deaminase.

As noted above, in some cases, a system of the present disclosure comprises a nucleic acid comprising a nucleotide sequence encoding a CRISPR-Cas effector polypeptide, or comprises a nucleic acid comprising a nucleotide sequence encoding a CRISPR-Cas effector fusion polypeptide. In some cases, the nucleic acid is present in an expressible construct, e.g., a recombinant expression vector. Suitable expression vectors include viral expression vectors (e.g. viral vectors based on vaccinia virus; poliovirus; adenovirus; SV40; herpes simplex virus; human immunodeficiency virus; a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, a recombinant expression vector is a recombinant adeno-associated virus (AAV) vector. In some cases, a recombinant expression vector is a recombinant lentivirus vector. In some cases, a recombinant expression vector is a recombinant retroviral vector.

Depending on the host/vector system utilized, any of a number of suitable transcription and translation control elements, including constitutive and inducible promoters, transcription enhancer elements, transcription terminators, etc. may be used in the expression vector.

In some cases, a nucleotide sequence encoding CRISPR-Cas effector polypeptide, or encoding a CRISPR-Cas effector fusion polypeptide, is operably linked to a transcriptional control element, e.g., a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the promoter is a neuron-specific promoter.

Suitable promoters include, but are not limited to, a CAG promoter (Miyazaki et al. (1989) Gene 79:269); a cytomegalovirus (CMV) promoter; a glutamate metabotropic receptor-6 (grm6) promoter (Cronin et al. (2014) EMBO Mol. Med. 6:1175); a Pleiades promoter (Portales-Casamar et al. (2010) Proc. Natl. Acad. Sci. USA 107:16589); a choline acetyltransferase (ChAT) promoter (Misawa et al. (1992) J. Biol. Chem. 267:20392); a vesicular glutamate transporter (V-glut) promoter (Zhang et al. (2011) Brain Res. 1377:1); a glutamic acid decarboxylase (GAD) promoter (Rasmussen et al. (2007) Brain Res. 1144:19; Ritter et al. (2016) J. Gene Med. 18:27); a cholecystokinin (CCK) promoter (Ritter et al. (2016) J. Gene Med. 18:27); a parvalbumin (PV) promoter; a somatostatin (SST) promoter; a neuropeptide Y (NPY) promoter; and a vasoactive intestinal peptide (VIP) promoter. Also suitable for use is an L7 promoter (Oberdick et al. (1990) Science 248:223), a thy-1 promoter, a recoverin promoter (Wiechmann and Howard (2003) Curr. Eye Res. 26:25); a calbindin promoter; and a beta-actin promoter. Suitable promoters include synthetic (non-naturally occurring) promoter/enhancer combinations.

Other suitable promoters include, for example, a gamma-synuclein (SNCG) promoter (e.g., Chaffiol et al. (2017) Mol. Ther. 25(11) 2546), a CBh promoter (e.g., Grey et al. (2011) Hum. Gene Ther. 22(9):1143-53), a miniCAG promoter (e.g., Grey et al. (2011) Hum. Gene Ther. 22(9):1143-53), a neurofilament heavy (NEFH) promoter (Millington-Ward et al. (2020) Sci. Rep. 10:16515), a G protein-coupled receptor kinase 1 (GRKI) promoter (e.g., Khani et al. (2007) Invest. Ophthalmol. Vis. Sci. 48(9):3954-61), a retinaldehyde-binding protein 1 (RLBP1) promoter (e.g., Choi et al. (2015) Mol. Ther. Methods Clin. Dev. 2: 15022; Vogel et al. (2007) Invest. Ophthalmol. Vis. Sci. 48, 3872-3877), a vitelliform muscular dystrophy-2 (VMD2) promoter (e.g., Conlon et al. (2013) Hum. Gene Ther. Clin. Dev. 24, 23-28), a synapsin I (Syn1) promoter (e.g., Kugler et al. (2003)), an enhSyn1 promoter (e.g., Hioki et al. (2007) Gene Ther. 14(11):872-82), and a neurofilament heavy (NEFH) promoter, or a functional fragment or variant thereof.

### Micro Poison Exon

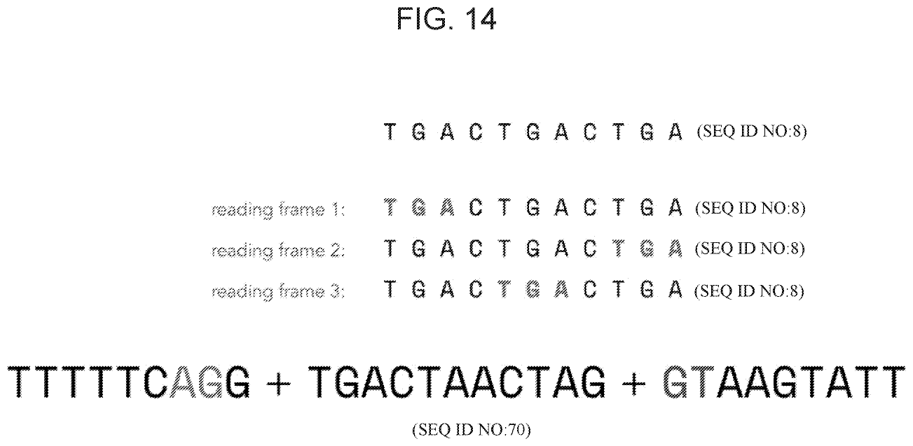

As noted above, a system of the present disclosure can include a poison exon insertion nucleotide sequence that provides for insertion of a poison exon into the target nucleic acid, wherein the poison exon insertion nucleotide sequence has a length of from about 21 nucleotides to about 150 nucleotides (e.g., from about 21 nucleotides to about 45 nucleotides, from about 21 nucleotides to about 50 nucleotides, from about 21 nucleotides to about 75 nucleotides, from about 21 nucleotides to about 100 nucleotides, from about 21 nucleotides to about 150 nucleotides) and comprises, from 5′ to 3′: i) a nucleotide sequence of from about 5 nucleotides to about 15 nucleotides in length and having a first consensus splice motif; ii) a nucleotide sequence of from about 11 nucleotides to about 15 nucleotides in length and comprising a stop codon in each reading frame; and iii) a nucleotide sequence of from about 5 nucleotides to about 15 nucleotides in length and having a second consensus splice motif. Such a poison exon is referred to as a “micro poison exon.” See, e.g., FIG. 14.

Examples of a micro poison exon sequence include, e.g.,

TTTTTCAGG(N)ₙ₁TGACTAACTAG(N)ₙ₂GTAAGTATT (SEQ ID NO:70), where N is any nucleotide and n1 and n2 are independently zero (0) or an integer from 1 to 25 (e.g., 1, 2, 3, 4, 5, 6, 7, 8 9, 10, 11-15, 15-20, or 20-25).

### Compositions

The present disclosure provides a composition comprising the components of a system of the present disclosure (e.g., as described above, where the components include, e.g., a) a CRISPR-Cas effector polypeptide or a fusion protein comprising a CRISPR-Cas effector polypeptide; b) a guide RNA; and, optionally, c) a donor nucleic acid. In some cases, the guide RNA comprises the one or more of a base modification, a sugar modification, and a backbone modification.

A composition of the present disclosure can include, in addition to the aforementioned components, one or more of: a lipid; a salt, e.g., NaCl, MgCl₂, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a nuclease inhibitor; glycerol; and the like.

The components of the composition may be present in a liposome. The components of the composition may be present within a particle, e.g., a nanoparticle, such as a lipid nanoparticle. The components of the composition may be present within a virus-like particle. In some cases, the lipid nanoparticle or the virus-like particle comprises a targeting moiety that provides for selective delivery of the composition to a particular organ, cell, or cell type.

The composition may comprise a pharmaceutically acceptable excipient, a variety of which are known in the art and need not be discussed in detail herein. Pharmaceutically acceptable excipients have been amply described in a variety of publications, including, for example, “Remington: The Science and Practice of Pharmacy”, 19^{th} Ed. (1995), or latest edition, Mack Publishing Co; A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

A pharmaceutical composition can comprise the aforementioned components, and a pharmaceutically acceptable excipient. In some cases, a subject pharmaceutical composition will be suitable for administration to a subject, e.g., will be sterile. For example, in some cases, a subject pharmaceutical composition will be suitable for administration to a human subject, e.g., where the composition is sterile and is free of detectable pyrogens and/or other toxins.

### Utility

A method of the present disclosure can be used for treating a disease caused by a toxic gain-of-function polypeptide in an individual. Thus, the present disclosure provides a method of for treating a disease caused by a toxic gain-of-function polypeptide in an individual, the method comprising reducing the level of an RNA transcript from a target nucleic acid encoding the toxic gain-of-function polypeptide, comprising modifying the nucleotide sequence of the target nucleic acid such that a spliced mRNA product of the modified target nucleic acid comprises a stop codon not present in a spliced mRNA product of the target nucleic acid (the unmodified target nucleic acid; the target nucleic acid before modification), wherein the spliced mRNA product undergoes nonsense-mediated mRNA decay, thereby reducing the level of the RNA transcript. By “reducing the level of the RNA transcript” is meant reduction in the number of RNA transcripts, which can lead to a reduction in the level of the polypeptide encoded by the RNA transcripts. The methods comprise administering to the individual a system of the present disclosure or a composition of the present disclosure.

A system or composition of the present disclosure can be administered to an individual in need thereof in any of a variety of ways and via any of a variety of routes of administration. The proteins and/or polynucleotides, and compositions comprising the proteins and/or polynucleotides described herein may be delivered to a target cell by any suitable means including, for example, by injection of proteins, via mRNA and/or using an expression construct (e.g., plasmid, lentiviral vector, AAV vector, Ad vector, etc.).

In some cases, a method of the present disclosure comprises administering to the individual a ribonucleoprotein (RNP) comprising a CRISPR-Cas effector polypeptide and a guide nucleic acid. In some cases, a method of the present disclosure comprises administering to the individual an RNP and a donor nucleic acid. In some cases, a method of the present disclosure comprises administering to the individual a composition comprising: a) a nucleic acid (e.g., an expressible construct, such as a recombinant expression vector) comprising a nucleotide sequence encoding a CRISPR-Cas effector polypeptide; and b) a guide RNA. In some cases, a method of the present disclosure comprises administering to the individual a nucleic acid (e.g., an expressible construct, such as a recombinant expression vector) comprising a first nucleotide sequence encoding a CRISPR-Cas effector polypeptide and a second nucleotide sequence encoding a guide RNA. In some cases, a method of the present disclosure comprises administering to the individual a composition comprising: a) first nucleic acid (e.g., an expressible construct, such as a recombinant expression vector) comprising a nucleotide sequence encoding a CRISPR-Cas effector polypeptide; and b) a second nucleic acid (e.g., an expressible construct, such as a recombinant expression vector) comprising a nucleotide sequence encoding a guide RNA.

In some cases, a method of the present disclosure comprises administering to the individual an RNP comprising a CRISPR-Cas effector fusion polypeptide and a guide nucleic acid. In some cases, a method of the present disclosure comprises administering to the individual an RNP and a donor nucleic acid. In some cases, a method of the present disclosure comprises administering to the individual a composition comprising: a) a nucleic acid (e.g., an expressible construct, such as a recombinant expression vector) comprising a nucleotide sequence encoding a CRISPR-Cas effector fusion polypeptide; and b) a guide RNA. In some cases, a method of the present disclosure comprises administering to the individual a nucleic acid (e.g., an expressible construct, such as a recombinant expression vector) comprising a first nucleotide sequence encoding a CRISPR-Cas effector fusion polypeptide and a second nucleotide sequence encoding a guide RNA. In some cases, a method of the present disclosure comprises administering to the individual a composition comprising: a) first nucleic acid (e.g., an expressible construct, such as a recombinant expression vector) comprising a nucleotide sequence encoding a CRISPR-Cas effector fusion polypeptide; and b) a second nucleic acid (e.g., an expressible construct, such as a recombinant expression vector) comprising a nucleotide sequence encoding a guide RNA.

As noted above, in some cases, a CRISPR-Cas effector polypeptide, a CRISPR-Cas fusion effector polypeptide, or a guide RNA, can be delivered in a vector. Any vector systems may be used including, but not limited to, plasmid vectors, retroviral vectors, lentiviral vectors, adenovirus vectors, poxvirus vectors; herpesvirus vectors and adeno-associated virus vectors, etc.

Conventional viral and non-viral based gene transfer methods can be used to introduce nucleic acids encoding a CRISPR-Cas effector polypeptide, a CRISPR-Cas fusion effector polypeptide, or a guide RNA into cells and target tissues. In certain embodiments, nucleic acids encoding a CRISPR-Cas effector polypeptide, a CRISPR-Cas fusion effector polypeptide, or a guide RNA are administered for in vivo or ex vivo. Non-viral vector delivery systems can also be used; where suitable non-viral vector delivery systems include DNA plasmids, naked nucleic acid, and nucleic acid complexed with a delivery vehicle such as a liposome or poloxamer. Viral vector delivery systems include DNA and RNA viruses, which can be episomal or can be integrated into the genome after delivery to the cell.

Methods of non-viral delivery of nucleic acids include electroporation, lipofection, microinjection, biolistics, virosomes, liposomes, immunoliposomes, polycation or lipid:nucleic acid conjugates, naked DNA, naked RNA, artificial virions, and agent-enhanced uptake of DNA. Sonoporation using, e.g., the Sonitron 2000 system can also be used for delivery of nucleic acids. In some cases, one or more nucleic acids are delivered as mRNA. In some cases, capped mRNAs are used to increase translational efficiency and/or mRNA stability.

Delivery can be to cells (ex vivo administration) or target tissues (in vivo administration).

In some cases, a system or a composition of the present disclosure is administered as a lipid:nucleic acid complex. The preparation of lipid:nucleic acid complexes, including targeted liposomes such as immunolipid complexes, is well known to one of skill in the art.

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis.

A system or a composition of the present disclosure can be administered via any of a variety of routes of administration, including, e.g., intracerebral, intracranial, intrathecal, intramuscular, intravenous, and the like.

A single dose or multiple doses of a system or a composition of the present disclosure can be administered to an individual in need thereof. The frequency of administration of the system or composition can vary depending on any of a variety of factors, e.g., severity of the symptoms, etc. For example, in some cases, a system or composition of the present disclosure is administered once per month, twice per month, three times per month, every other week (qow), once per week (qw), once every two weeks, once every three weeks, once every four weeks, twice per week (biw), three times per week (tiw), four times per week, five times per week, six times per week, every other day (qod), daily (qd), twice a day (qid), or three times a day (tid).

Diseases that can be treated using a method of the present disclosure include any disease in which a toxic gain-of-function polypeptide is produced and gives rise to the disease. Such diseases include those in which the toxic gain-of-function polypeptide results from repeat expansions, deletions, or point mutations, where the repeat expansions, deletions, or point mutations generally occur in the coding region of a genome.

Diseases that can be treated using a method of the present disclosure include trinucleotide repeat expansion diseases, tetranucleotide repeat expansion diseases, and hexanucleotide repeat expansion diseases, and any other disease in which a toxic gain-of-function polypeptide is produced and gives rise to the disease. Examples include, e.g., a CAG trinucleotide repeat expansion (where such repeat expansions give rise to Huntington's disease, spinal and bulbar muscular atrophy, dentatorubral-pallidoluysian atrophy, and several spinocerebellar ataxias); CTG trinucleotide repeat expansions (where such repeat expansions give rise to myotonic dystrophy 1, spinocerebellar ataxia type 8, Fuchs corneal dystrophy, and Huntington's disease-like 2); GAA trinucleotide repeats (where such trinucleotide repeats give rise to Friedreich's ataxia); GGGGCC hexanucleotide repeat expansions (where such hexanucleotide repeat expansions give rise to C9orf72 frontotemporal dementia and amyotrophic lateral sclerosis); and the like. See, e.g., Paulson (2018) Handbook of Clinical Neurology 147:105.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A method of reducing the level of an RNA transcript from a target nucleic acid, the method comprising modifying the nucleotide sequence of the target nucleic acid such that a spliced mRNA product of the modified target nucleic acid comprises a stop codon that is not present in a spliced mRNA transcript of the unmodified target nucleic acid and wherein, as a result of the stop codon, the spliced mRNA product undergoes nonsense-mediated mRNA decay, thereby reducing the level of the RNA transcript.

Aspect 2. The method of aspect 1, wherein the spliced mRNA product of the modified target nucleic acid comprises a stop codon induced by a frameshift caused by the inclusion of an exon not present in the spliced mRNA of the unmodified target nucleic acid.

Aspect 3. The method of aspect 2, wherein said modifying comprises modifying the nucleotide sequence in the target nucleic acid to include a splice dinucleotide that is not present in the unmodified target nucleic acid, optionally wherein the splice dinucleotide is an AG dinucleotide or a GT dinucleotide.

Aspect 4. The method of aspect 2, wherein said modifying comprises modifying a polypyrimidine tract that is immediately 5′ of an endogenous AG dinucleotide, generating a modified AG dinucleotide-containing sequence, such that a spliceosome in a cell would recognize the modified AG dinucleotide-containing sequence as a splice acceptor dinucleotide.

Aspect 5. The method of aspect 2, wherein said modifying comprises modifying a consensus sequence within 10 nucleotides of an endogenous GT dinucleotide, generating a modified GT dinucleotide-containing sequence, such that a spliceosome in a cell would recognize the modified GT dinucleotide-containing sequence as a splice donor dinucleotide.

Aspect 6. The method of aspect 1, comprising modifying an exon of the target nucleic acid such that the modified target nucleic acid includes a stop codon that is not present in an exon of the unmodified target nucleic acid.

Aspect 7. The method of any one of aspects 1-6, wherein modifying comprises contacting the target nucleic acid with a CRISPR-Cas effector polypeptide and a guide nucleic acid, wherein the guide nucleic acid comprises: i) a targeting region that comprises a nucleotide sequence that binds to a target sequence in the target nucleic acid; and ii) a protein-binding region that binds to the CRISPR-Cas effector polypeptide.

Aspect 8. The method of aspect 6, further comprising contacting the target nucleic acid with a donor nucleic acid that comprises a nucleotide sequence that includes the modification that results in the stop codon.

Aspect 9. The method of aspect 7 or aspect 8, wherein the CRISPR-Cas effector polypeptide is a type II CRISPR-Cas effector polypeptide, a type III CRISPR-Cas effector polypeptide, a type IV CRISPR-Cas effector polypeptide, a type V CRISPR-Cas effector polypeptide, or a type VI CRISPR-Cas effector polypeptide.

Aspect 10. The method of any one of aspects 1-9, wherein modifying comprises contacting the target nucleic acid with: a) a fusion CRISPR-Cas effector polypeptide comprising: i) a CRISPR-Cas effector polypeptide; and ii) one or more heterologous polypeptides; and b) a guide nucleic acid, wherein the guide nucleic acid comprises: i) a targeting region that comprises a nucleotide sequence that binds to a target sequence in the target nucleic acid; and ii) a protein-binding region that binds to the CRISPR-Cas effector polypeptide.

Aspect 11. The method of aspect 10, wherein the one or more heterologous polypeptides comprises a reverse transcriptase.

Aspect 12. The method of aspect 11, wherein the CRISPR-Cas effector polypeptide is a nickase.

Aspect 13. The method of aspect 10, wherein the one or more heterologous polypeptides comprises a cytidine deaminase.

Aspect 14. The method of aspect 10, wherein the one or more heterologous polypeptides comprises an adenine deaminase.

Aspect 15. The method of aspect 13 or aspect 14, wherein the CRISPR-Cas effector polypeptide is a nickase.

Aspect 16. The method of any one of aspects 1-15, wherein the target nucleic acid is present in a eukaryotic cell.

Aspect 17. The method of aspect 16, wherein the eukaryotic cell is in vitro.

Aspect 18. The method of aspect 16, wherein the eukaryotic cell is in vivo.

Aspect 19. The method of any one of aspects 16-18, wherein the eukaryotic cell comprises a disease-associated mutation-containing allele and a corresponding wild-type allele that does not comprise the disease-associated mutation, and wherein the disease-associated mutation-containing allele comprises the target nucleic acid.

Aspect 20. The method of aspect 19, wherein the disease-associated mutation-containing allele comprises a single nucleotide polymorphism (SNP) that is not present in the corresponding wild-type allele, and wherein the method comprises contacting the target nucleic acid with: a) a CRISPR-Cas effector polypeptide or a fusion polypeptide comprising a CRISPR-Cas effector polypeptide; and b) a guide nucleic acid, wherein the guide nucleic acid comprises: i) a targeting region that comprises a nucleotide sequence that binds to a target sequence in the target nucleic acid, wherein the target sequence comprises the SNP; and ii) a protein-binding region that binds to the CRISPR-Cas effector polypeptide.

Aspect 21. The method of any one of aspects 1-20, wherein the target nucleic acid encodes a toxic gain-of-function polypeptide.

Aspect 22. The method of aspect 21, wherein the target nucleic acid comprises a trinucleotide, a tetranucleotide, or a hexanucleotide repeat expansion.

Aspect 23. A method for treating a disease caused by a toxic gain-of-function polypeptide in an individual, the method comprising reducing the level of an RNA transcript from a target nucleic acid encoding the toxic gain-of-function polypeptide, comprising modifying the nucleotide sequence of the target nucleic acid such that a spliced mRNA product of the modified target nucleic acid comprises an exon that comprises a stop codon not present in a spliced mRNA product of the unmodified target nucleic acid, wherein the spliced mRNA product undergoes nonsense-mediated mRNA decay, thereby reducing the level of the RNA transcript and treating the disease.

Aspect 24. The method of aspect 23, wherein the disease is a trinucleotide repeat expansion disease.

Aspect 25. The method of aspect 23, wherein the disease is a tetranucleotide repeat expansion disease.

Aspect 26. The method of aspect 23, wherein the disease is a hexanucleotide repeat expansion disease.

Aspect 27. A system for reducing the level of an RNA transcript of a target nucleic acid in a eukaryotic cell, the system comprising:
a) a CRISPR-Cas effector polypeptide, or a nucleic acid comprising a nucleotide sequence encoding a CRISPR-Cas effector polypeptide;
b) a guide RNA comprising i) a targeting region that comprises a nucleotide sequence that binds to a target sequence in the target nucleic acid; and ii) a protein-binding region that binds to the CRISPR-Cas effector polypeptide; and
c) a donor nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′:
i) a nucleotide sequence of from about 5 nucleotides to about 15 nucleotides in length and having a first consensus splice motif;
ii) a nucleotide sequence of from about 11 nucleotides to about 15 nucleotides in length and comprising a stop codon in each reading frame; and
iii) a nucleotide sequence of from about 5 nucleotides to about 15 nucleotides in length and having a second consensus splice motif.

Aspect 28. The system of aspect 27, wherein the CRISPR-Cas effector polypeptide is a type II CRISPR-Cas effector polypeptide, a type III CRISPR-Cas effector polypeptide, a type IV CRISPR-Cas effector polypeptide, a type V CRISPR-Cas effector polypeptide, or a type VI CRISPR-Cas effector polypeptide.

Aspect 29. A system for reducing the level of an RNA transcript from a target nucleic acid in a eukaryotic cell, the system comprising:
a) a CRISPR-Cas effector fusion polypeptide, or a nucleic acid comprising a nucleotide sequence encoding a CRISPR-Cas effector fusion polypeptide, wherein the CRISPR-Cas effector fusion polypeptide comprises: i) a CRISPR-Cas effector polypeptide; and ii) one or more heterologous fusion partners;
b) a guide RNA comprising i) a targeting region that comprises a nucleotide sequence that binds to a target sequence in the target nucleic acid; ii) a protein-binding region that binds to the CRISPR-Cas effector polypeptide; and ii) a nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′:
i) a nucleotide sequence of from about 5 nucleotides to about 15 nucleotides in length and having a first consensus splice motif;
ii) a nucleotide sequence of from about 11 nucleotides to about 15 nucleotides in length and comprising a stop codon in each reading frame; and
iii) a nucleotide sequence of from about 5 nucleotides to about 15 nucleotides in length and having a second consensus splice motif.

Aspect 30. The system of aspect 29, wherein the one or more heterologous polypeptides comprises a reverse transcriptase and wherein the guide RNA comprises a primer binding nucleotide sequence.

Aspect 31. The system of aspect 29 or aspect 30, wherein the CRISPR-Cas effector polypeptide is a nickase.

Aspect 32. The system of any one of 29-31, wherein the CRISPR-Cas effector polypeptide is a type II CRISPR-Cas effector polypeptide, a type III CRISPR-Cas effector polypeptide, a type IV CRISPR-Cas effector polypeptide, a type V CRISPR-Cas effector polypeptide, or a type VI CRISPR-Cas effector polypeptide.

Aspect 33. A composition comprising:
a) a CRISPR-Cas effector polypeptide, or a nucleic acid comprising a nucleotide sequence encoding a CRISPR-Cas effector polypeptide;
b) a guide RNA comprising i) a targeting region that comprises a nucleotide sequence that binds to a target sequence in the target nucleic acid; and ii) a protein-binding region that binds to the CRISPR-Cas effector polypeptide; and
c) a donor nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′:
i) a nucleotide sequence of from about 5 nucleotides to about 15 nucleotides in length and having a first consensus splice motif;
ii) a nucleotide sequence of from about 11 nucleotides to about 15 nucleotides in length and comprising a stop codon in each reading frame; and
iii) a nucleotide sequence of from about 5 nucleotides to about 15 nucleotides in length and having a second consensus splice motif.

Aspect 34. A composition comprising:
a) a CRISPR-Cas effector fusion polypeptide, or a nucleic acid comprising a nucleotide sequence encoding a CRISPR-Cas effector fusion polypeptide, wherein the CRISPR-Cas effector fusion polypeptide comprises: i) a CRISPR-Cas effector polypeptide; and ii) one or more heterologous fusion partners;
b) a guide RNA comprising i) a targeting region that comprises a nucleotide sequence that binds to a target sequence in the target nucleic acid; ii) a protein-binding region that binds to the CRISPR-Cas effector polypeptide; and ii) a nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′:
i) a nucleotide sequence of from about 5 nucleotides to about 15 nucleotides in length and having a first consensus splice motif;
ii) a nucleotide sequence of from about 11 nucleotides to about 15 nucleotides in length and comprising a stop codon in each reading frame; and
iii) a nucleotide sequence of from about 5 nucleotides to about 15 nucleotides in length and having a second consensus splice motif.

Aspect 35. The composition of aspect 34, wherein the one or more heterologous polypeptides comprises a reverse transcriptase and wherein the guide RNA comprises a primer binding nucleotide sequence.

Aspect 36. The composition of aspect 34, wherein the one or more heterologous polypeptides comprises a cytidine deaminase or an adenosine deaminase.

Aspect 37. A composition of any one or aspects 33-36, comprising one or more of a lipid, a protease inhibitor, a nuclease inhibitor, a salt, a divalent cation, and a buffer.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

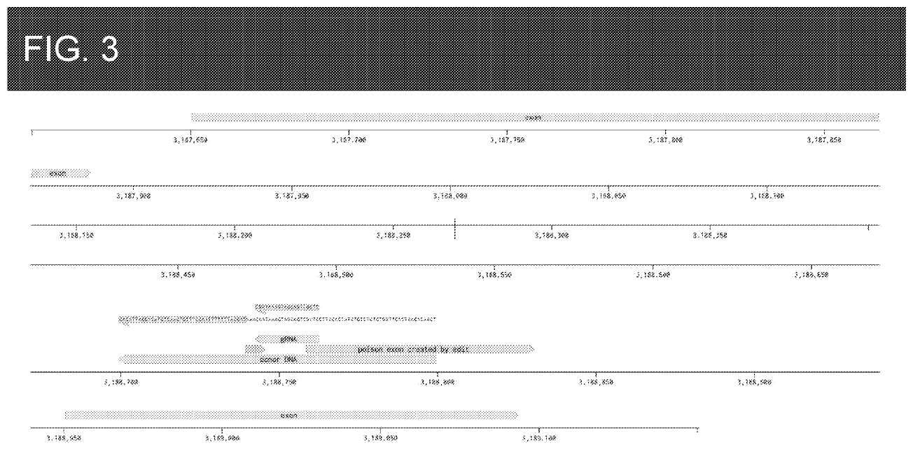

The SH-SY5Y cell line is a human neuroblastoma cell line that express huntingtin from the HTT gene. To test whether a poison exon could be introduced into the HTT gene, SH-SY5Y cells were transfected with a Cas9 polypeptide, a guide RNA, and a donor DNA, as depicted schematically in FIG. 3. RNA was collected from the edited cells and sequenced to evaluate HTT splicing, i.e., to test whether a new RNA isoform (spliced RNA transcript) was generated that included a premature stop codon as a result of introduction of the poison exon. Whether the new RNA isoform was targeted for degradation via nonsense-mediated decay (NMD) was also tested. The levels of the new RNA isoform produced in the presence or absence of the NMD inhibitor cycloheximide (CHX) were assayed to determine whether the new isoform of the RNA was targeted for degradation via NMD.

### Materials and Methods

### SHSY5Y Editing

SHSY5Y cells were obtained from the UC Berkeley Cell Culture facility and cultured at 37° C. and 5% CO₂ in SHSY5Y media (1X DMEM Glutamax-I with 10% Fetal Bovine Serum (FBS), 1% MEM non-essential amino acids (NEAA) and 1% Sodium Pyruvate). Alt-R CRISPR-Cas9 crRNA guide 3 (5′-CTCGCTGAGGATGAAATGGC-3′; SEQ ID NO:75), Alt-R tracrRNA and Alt-R homology-directed repair (HDR) donor oligonucleotide (“oligo”) containing the desired edit (5′-TGGACACAGTCACTTGGTCTCTCTGTGTATCACCTTCCTCGCTGAGGATGAAATAACAAATA GCATTTTTTTAAGCTTTGTGAACTGTACACCATTACAC-3′; SEQ ID NO:88) were ordered from Integrated DNA Technologies (IDT). crRNA and tracrRNA were each resuspended in IDT nuclease-free duplex buffer to a 160 μM concentration. To assemble guide RNA, 1:1 volume of each crRNA and tracrRNA were mixed and incubated at 37° C. for 30 min. For ribonucleoprotein complex (RNP) assembly, a 1:1 volume of 40 μM Cas9-NLS (QB3 Macrolab UC Berkeley) to guide RNA were mixed and incubated at 37° C. for 15 min. SHSY5Y cells (2×10⁵) were resuspended in 18 μl of SF nucleofector solution with supplement (Lonza) and mixed with 2.5 μl of RNP and 0.8 μl of 100 μM HDR donor oligo. SHSY5Y cells were nucleofected using Amaxa 4D-Nucleofector (Lonza) with program CA-137 and incubated at room temperature for 10 min before addition of SHSY5Y media and plating. After 48 hours in culture, genomic DNA was extracted from 1×10⁵ cells using 50 μl of QuickExtract DNA Extraction Solution (Lucigen). Remaining cells were plated for expansion and passaged when they reached ˜80% confluency. Cells in QuickExtract were vortexed and incubated at 65° C. for 20 min and 95° C. for 20 min. To prepare samples for Next Generation Sequencing (NGS), polymerase chain reaction (PCR) was performed using PrimeSTAR GXL SP DNA Polymerase (Takara Bio Inc) with primers containing a 13-nucleotide NGS tag at the 5′ end (forward primer: 5′-(GCTCTTCCGATCT)TGAAGCAGCTGGCTAAAATTGA-3′ (SEQ ID NO:89) and reverse primer: 5′-(GCTCTTCCGATCT)TAAAGTGAAATGCAGAGCGCC-3′ (SEQ ID NO:90)). PCR products were purified using QIAquick PCR purification kit (Qiagen) and submitted to the Innovative Genomics Institute NGS Core (UC Berkeley) for partial amplicon library preparation and sequencing with MiSeq Kit v2 (300 cycles) (Illumina). NGS results were analyzed using CRISPResso2 (at https:(double forward slash)crispresso(dot)pinellolab(dot)partners(dot)org) to determine HDR editing efficiency.

### Poison Exon Inclusion Assay

SHSY5Y cells (non-edited or HDR edited) were plated at a density of 3×10⁵ cells per well of 6-well plate and cultured overnight in SHSY5Y media. The next day, media was removed and replaced with SHSY5Y media containing either mock treatment (DMSO only) or cycloheximide treatment (CHX) at concentrations of 10 μg/ml CHX or 30 μg/ml CHX to inhibit nonsense-mediated decay (NMD). After 6 hours of incubation at 37° C. and 5% CO₂, SHSY5Y cells were washed with sterile phosphate buffered saline (PBS) followed by trypsinization with 0.05% trypsin/EDTA for 3 min at 37° C. SHSY5Y media was added and cells were collected and centrifuged at 1500 rpm for 5 min. Supernatant was removed, and RNA was extracted and purified from pelleted cells using miRNeasy micro kit (Qiagen). Extracted RNA was used for reverse transcription using Maxima First Strand cDNA Synthesis kit (Thermo Fisher Scientific). PCR amplification of cDNA was performed using Taq2X Master Mix (New England BioLabs) using primers in the flanking exons. The forward primer was 5′-TTCTGGCCATTTTGAGGGTTCT-3′ (SEQ ID NO:91) and the reverse primer was 5′-TGCTCACTCATTTCCACCTTCA-3′ (SEQ ID NO:92). PCR products were purified using QIAquick PCR purification kit (Qiagen) and run on an Egel EX 1% agarose (Invitrogen). Expected PCR product sizes were 274 bp for regular exons and 346 bp if there was inclusion of the predicted poison exon. Band intensity was quantified using Fiji (ImageJ) software (imagej.net/software/fiji/).

### Results

A poison exon was introduced into the HTT locus of SHSY5Y cells via Cas9-mediated HDR. After gene editing, the edited SHSY5Y cells were either mock treated (DMSO only) or CHX treated at 10 g/ml or 30 g/ml for 6 hours. After the treatment, RNA was extracted and analyzed. The RNA was subjected to RT-PCR. The PCR products were analyzed via gel electrophoresis. The expected PCR product sizes were 274 bp for regular exons (transcripts from non-edited HTT alleles) and 346 bp for edited alleles (transcripts of HTT alleles that included the poison exon).

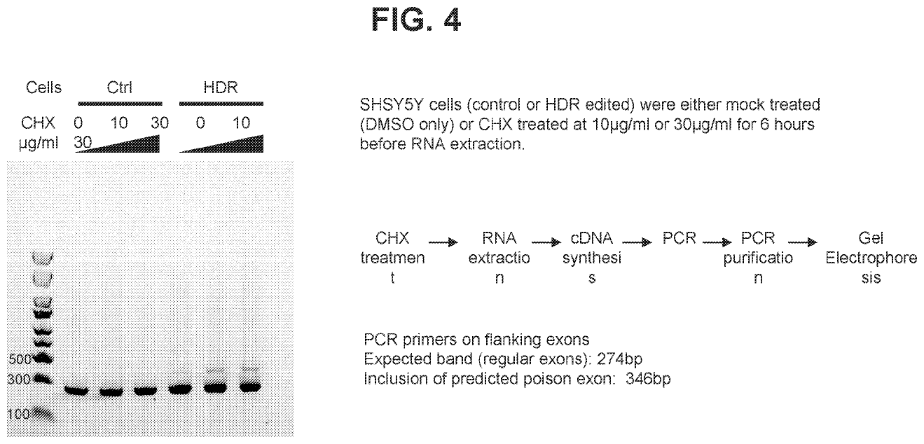

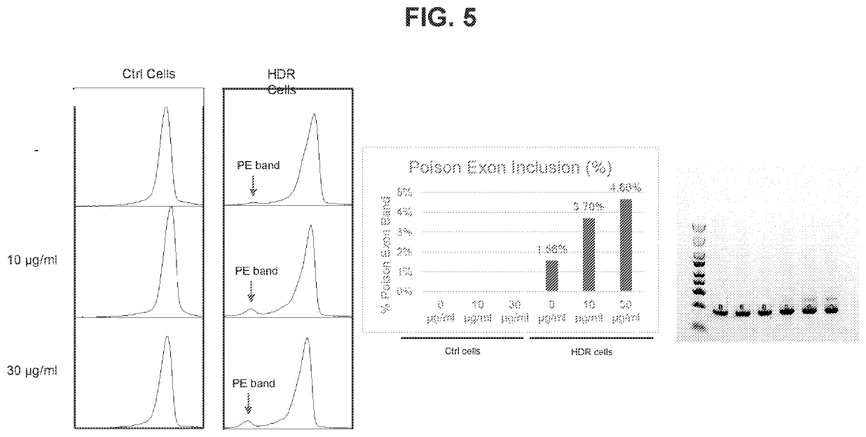

The results are shown in FIG. 4 and FIG. 5. Analysis showed that HDR editing efficiency was approximately 25%. As shown in FIG. 4 and FIG. 5, PCR of HTT mRNA from edited cells resulted in a second, longer, product that included the poison exon, along with the normal HTT product found in both samples. When NMD was inhibited by a high concentration of CHX, this longer product was shown to be ˜5% of the total. With NMD active, its abundance was reduced to 1.5%, indicating substantial degradation of the poison exon mRNA. Accounting for the HDR editing efficiency, the results show that mRNA from the edited locus was substantially spliced to include the poison exon, and that this mRNA was indeed degraded by NMD, reducing total HTT mRNA as intended.

### Example 2

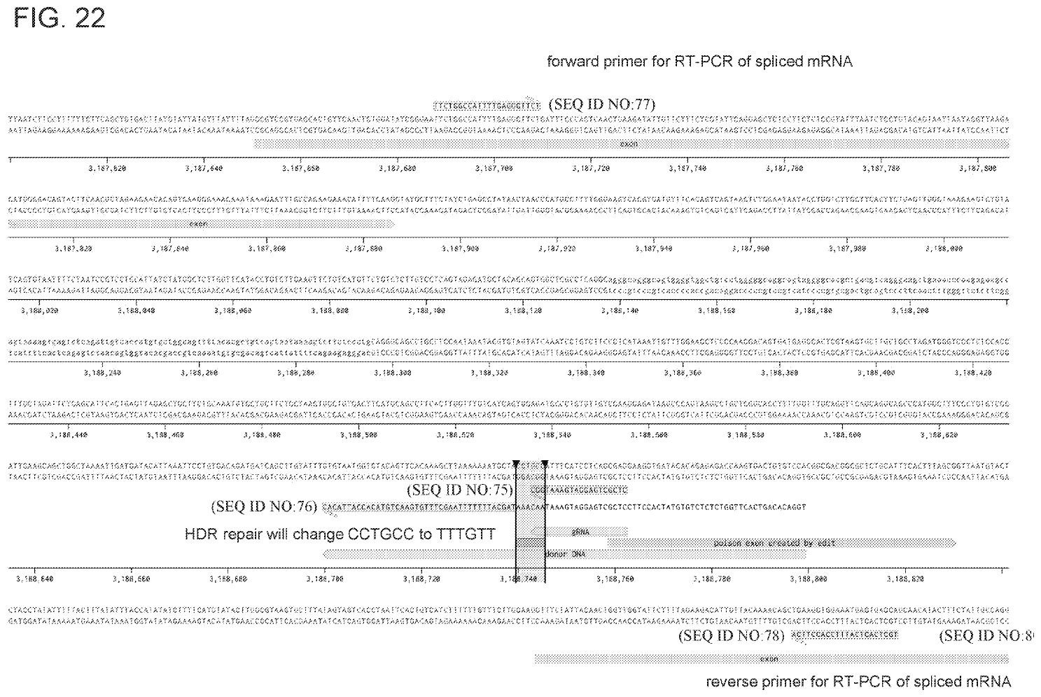

To test whether predicted base edits could introduce a poison exon into the HTT gene, HCT116 cells were nucleofected with a Cas9 polypeptide, a guide RNA, and a single stranded donor DNA comprising the predicted base edit as depicted schematically in FIG. 22. Following nucleofection, clonal cell lines were generated and sequenced to identify clonal cell lines with the intended edit. To test whether a new RNA isoform (spliced RNA transcript) was generated that included a premature stop codon and whether the newly generated RNA isoform was targeted for degradation via nonsense-mediated decay (NMD), the levels of the new RNA isoform produced in the presence or absence of the NMD inhibitor cycloheximide (CHX) was assayed.

### Materials and Methods

### HCT116 Editing for Predicted Base Editing Outcome

HCT116 cells were cultured in DMEM with 10% FBS. HCT116 cells were nucleofected using Amaxa 4D-Nucleofector with SE solution (Lonza) and program EN113 to deliver Cas9-NLS protein (QB3 Macrolab UC Berkeley) complexed with corresponding guide (crRNA:tracrRNA from IDT) and a single stranded donor DNA (IDT). To test the predicted results of base editing, guide SNP3g3 (5′-CTCGCTGAGGATGAAATGGC-3′; SEQ ID NO:75) and SNP3_HDR_donor (5′-GTGTAATGGTGTACAGTTCACAAAGCTTAAAAAAATGCTATTTGTTATTTCATCCTCAGCGA GGAAGGTGATACACAGAGAGACCAAGTGACTGTGTCCA-3′; SEQ ID NO:76) were used. After nucleofection, cells were plated on media with AltR HDR Enhancer V2 (IDT) and expanded. Clonal lines were generated from bulk edited population and tested using Sanger sequencing (as described above) to identify cells with the intended edit, either homozygous or heterozygous.

### HCT116 Poison Exon Inclusion Assay for Predicted Base Editing Outcome

Clonal lines were treated with cycloheximide for 6 hours to inhibit NMD, followed by RNA extraction with Direct-Zol RNA Miniprep kit (Zymo) and reverse transcription with Maxima First Strand cDNA synthesis kit (Themo Fisher Scientific). PCR amplification of cDNA was performed using OneTaq2X Master Mix (New England BioLabs) with primers in the flanking exons (SNP3EX FWD 5′-TTCTGGCCATTTTGAGGGTTCT-3′; SEQ ID NO:77 and SNP3EX REV 5′-TGCTCACTCATTTCCACCTTCA-3′; SEQ ID NO:78). Splicing was assayed by running PCR products on a 1.2% Egel with SYBR safe (Invitrogen). Expected PCR product sizes were 274 bp for regular exons and 346 bp or 352 bp if there was inclusion of the predicted poison exon. Band intensity was quantified using Fiji (ImageJ) software (imagej.net/software/fiji/).

### Results

A poison exon was introduced into the HTT locus of HCT116 cells via Cas9-mediated HDR. After gene editing and selection of clones with homozygous and heterozygous editing, the edited HCT116 cells were either mock treated (DMSO only) or CHX treated for 6 hours. After the treatment, RNA was extracted and analyzed. The RNA was subjected to RT-PCR. The PCR products were analyzed via gel electrophoresis. The expected PCR product sizes were 274 bp for regular exons (transcripts from non-edited HTT alleles) and 346 bp or 352 bp for edited alleles (transcripts of HTT alleles that included the poison exon).

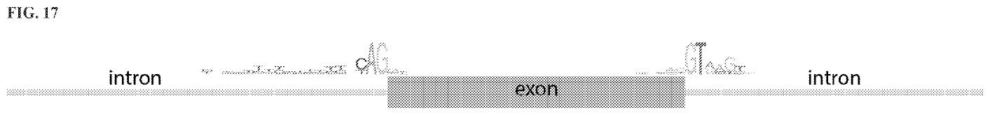

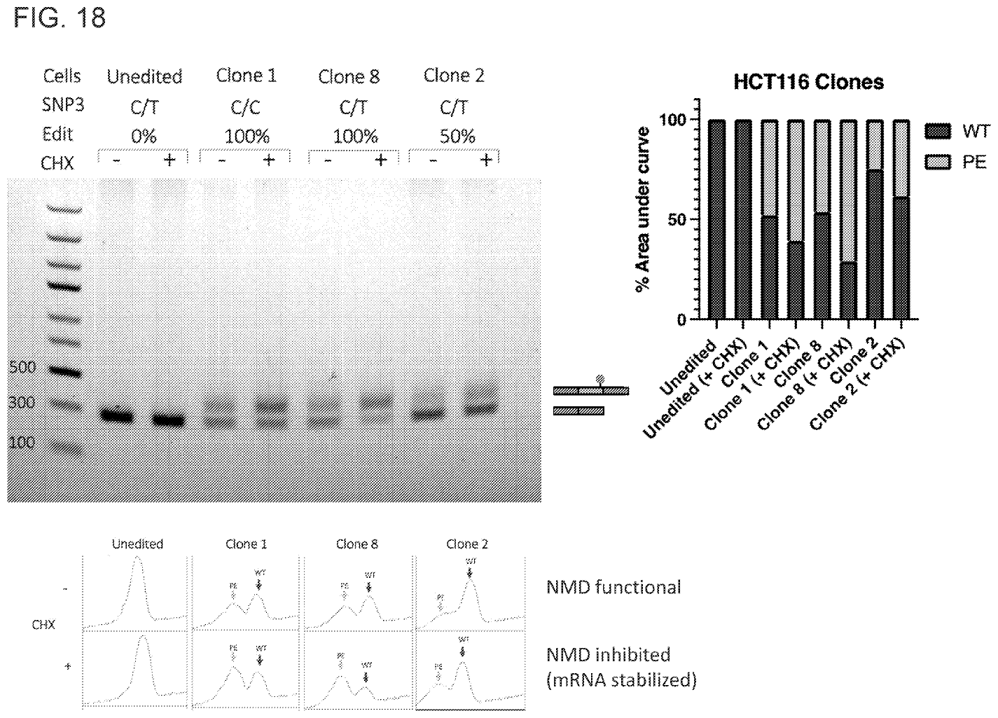

The results are shown in FIG. 18. As shown in FIG. 18, PCR of HTT mRNA from edited cells resulted in a second, longer, product that included the poison exon, along with the normal HTT product found in both samples. In homozygously edited cells, when NMD was inhibited by CHX, this longer product was shown to be ˜60%-75% of the total mRNA produced from the HTT gene. With NMD active, its abundance was reduced to ˜50%, indicating substantial degradation of the poison exon mRNA. In heterozygously edited cells, with only one copy of HTT harboring the edit, the longer product was shown to be ˜40% of the mRNA when NMD was inhibited and ˜25% of the mRNA when NMD was active. The results show that the majority of mRNA from the edited locus was spliced to include the poison exon, and that this mRNA was indeed degraded by NMD, reducing total HTT mRNA as intended.

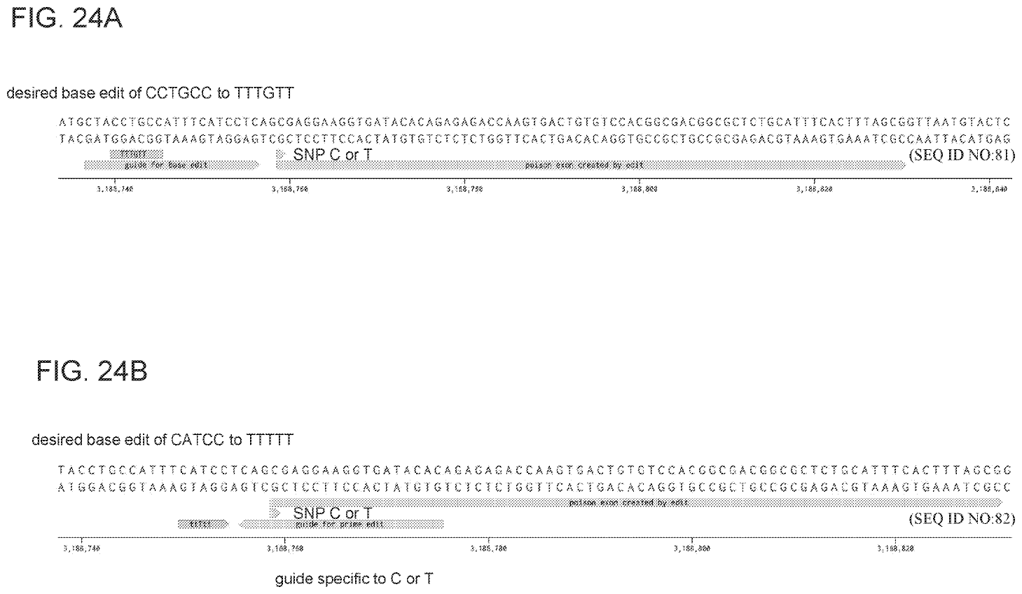

A schematic depiction of the use of a CRISPR-Cas guide RNA and a base editor to introduce a poison exon, or to introduce a mutation that gives rise to a poison exon, in a target nucleic acid is depicted in FIG. 24A.

### Example 3

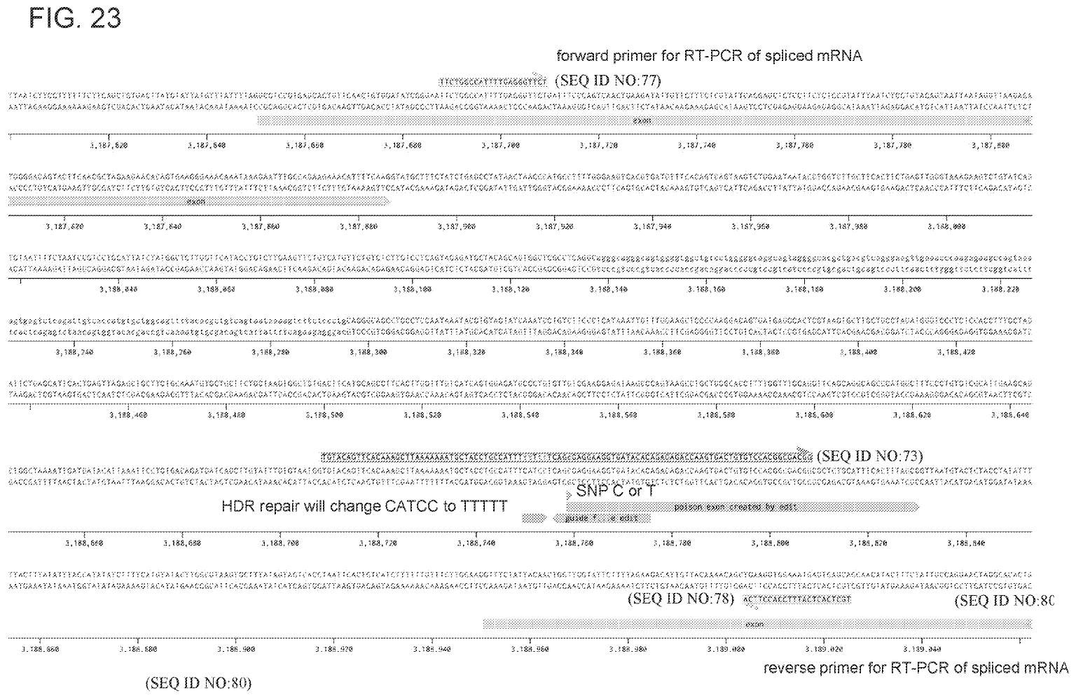

To test whether prime editing can be targeted in an allele-specific manner, HCT116 cells heterozygous at the target locus were nucleofected with a Cas9 polypeptide, an allele-specific guide, and a single stranded allele-specific donor DNA comprising the predicted prime edit as depicted schematically in FIG. 23. DNA was collected from the edited cells and sequenced by next generation sequencing (NGS) to evaluate the allele specificity of the intended edit. To test whether a new allele-specific RNA isoform (spliced RNA transcript) was generated that included a premature stop codon and whether the newly generated allele-specific RNA isoform was targeted for degradation via nonsense-mediated decay (NMD), the levels of the new allele-specific RNA isoform produced in the presence or absence of the NMD inhibitor cycloheximide (CHX) was assayed.

### Materials and Methods

### HCT116 Confirmation of Heterozygosity at SNP3 (Rs6844859)

HCT116 cells were cultured in DMEM with 10% FBS. DNA was extracted from HCT116 cells using QuickExtract (Lucigen) and the region of interest was PCR amplified with SNP3 FWD (5′-CGAAGGAGATAAGCCCAGTAAG-3′; SEQ ID NO:83) and SNP3 REV (5′-GAGACACTCCAGGAAGACTAATG-3′; SEQ ID NO:84) primers using OneTaq2X Master Mix. Product was purified with QIAquick PCR purification kit (Qiagen) and Sanger sequenced to confirm cells were heterozygous at SNP3 (rs6844859).

### Allele Selectivity of Potential Prime Editing Guides

HCT116 cells were nucleofected with Cas9-NLS protein and either SNP3PE_C_guide (5′-GTGTATCACCTTCCTCGCTG-3′; SEQ ID NO:85) or SNP3PE_T_guide (5′-GTGTATCACCTTCCTCACTG-3′; SEQ ID NO:86). After 2 days, DNA was extracted from cells using QuickExtract. PCR was performed using PrimeSTAR GXL DNA Polymerase (Takara Bio) with NGS primers (SNP3 HDR NGS FWD and SNP3 HDR NGS REV). PCR products were purified using QIAquick PCR purification kit and submitted to the Innovative Genomics Institute NGS Core (UC Berkeley) for library preparation and sequencing with MiSeq 2×150 bp (Illumina). NGS results were analyzed using CRISPResso2 (at https:(double forward slash)crispresso(dot)pinellolab(dot)partners(dot)org).

### HCT116 Editing for Predicted Prime Editing Outcome

HCT116 cells were nucleofected using Amaxa 4D-Nucleofector with SE solution (Lonza) and program EN113 to deliver Cas9-NLS protein (QB3 Macrolab UC Berkeley) complexed with corresponding guide (crRNA:tracrRNA from IDT) and a single stranded donor DNA (IDT). To test the predicted results of prime editing of either the C or the T SNP allele, guides specific to each SNP variant were used: SNP3PE_C_guide (5′-GTGTATCACCTTCCTCGCTG-3′; SEQ ID NO:85) with SNP3PE_C_donor (5′-TGTACAGTTCACAAAGCTTAAAAAAATGCTACCTGCCATTTttTttTCAGcGAGGAAGGTGATA CACAGAGAGACCAAGTGACTGTGTCCACGGCGACGG-3′; lower-case letters represent the intended changes and the SNP position; SEQ ID NO:73) or SNP3PE_T_guide (5′-GTGTATCACCTTCCTCACTG-3′; SEQ ID NO:86) with SNP3PE_T_donor (5′-TGTACAGTTCACAAAGCTTAAAAAAATGCTACCTGCCATTTttTttTCAGTGAGGAAGGTGATA CACAGAGAGACCAAGTGACTGTGTCCACGGCGACGG-3′; lower-case letters represent the intended changes; SEQ ID NO:87).

After nucleofection, cells were plated on media with AltR HDR Enhancer V2 (IDT) and expanded. Clonal lines were generated from bulk edited population and tested using Sanger sequencing (as described above) to identify cells with the intended edit, either homozygous or heterozygous.

### HCT116 Poison Exon Inclusion Assay for Predicted Prime Editing Outcome

Clonal lines were treated with cycloheximide for 6 hours, followed by RNA extraction with Direct-Zol RNA Miniprep kit (Zymo) and reverse transcription with Maxima First Strand cDNA synthesis kit (Themo Fisher Scientific). PCR amplification of cDNA was performed using OneTaq2X Master Mix (New England BioLabs) with primers in the flanking exons (SNP3EX FWD 5′-TTCTGGCCATTTTGAGGGTTCT-3′; SEQ ID NO:77 and SNP3EX REV 5′-TGCTCACTCATTTCCACCTTCA-3′; SEQ ID NO:78). Splicing was assayed by running PCR products on a 1.2% Egel with SYBR safe (Invitrogen). Expected PCR product sizes were 274 bp for regular exons and 346 bp or 352 bp if there was inclusion of the predicted poison exon. Band intensity was quantified using Fiji (ImageJ) software (imagej.net/software/fiji/).

### Results

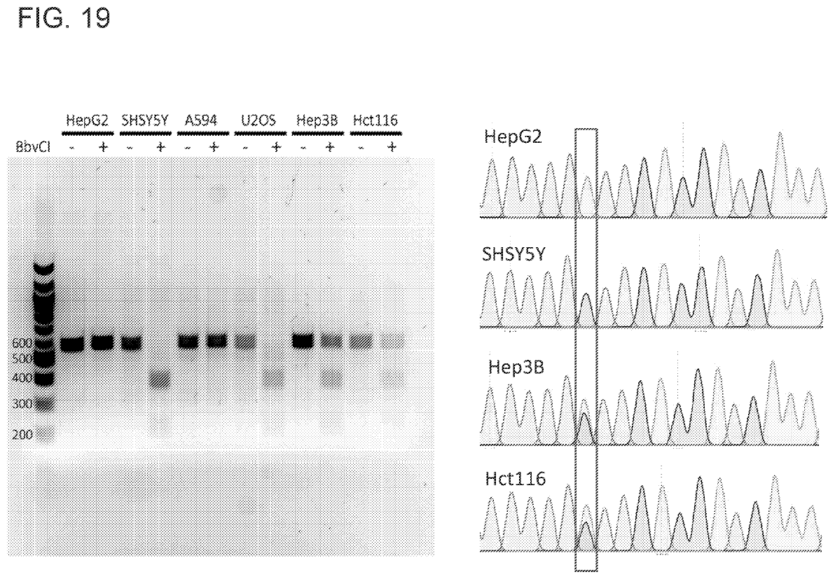

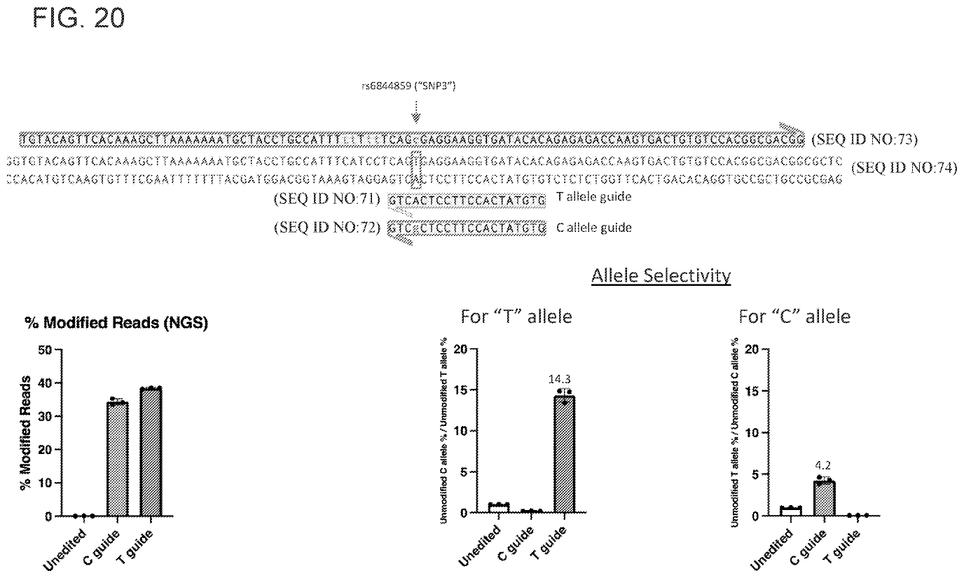

The HTT locus of HCT116 cells was shown to be heterozygous for the C and T alleles at SNP3 (rs6844859) (FIG. 19). Guides to introduce a poison exon into the HTT locus of HCT116 cells showed allele selectivity (FIG. 20).

A poison exon was introduced into the HTT locus of HCT116 cells via Cas9-mediated HDR, mimicking the predicted outcome of prime editing. After gene editing and selection of clones with homozygous and heterozygous editing, the edited HCT116 cells were either mock treated (DMSO only) or CHX treated for 6 hours. After the treatment, RNA was extracted and analyzed. The RNA was subjected to RT-PCR. The PCR products were analyzed via gel electrophoresis. The expected PCR product sizes were 274 bp for regular exons (transcripts from non-edited HTT alleles) and 346 bp or 352 bp for edited alleles (transcripts of HTT alleles that included the poison exon).

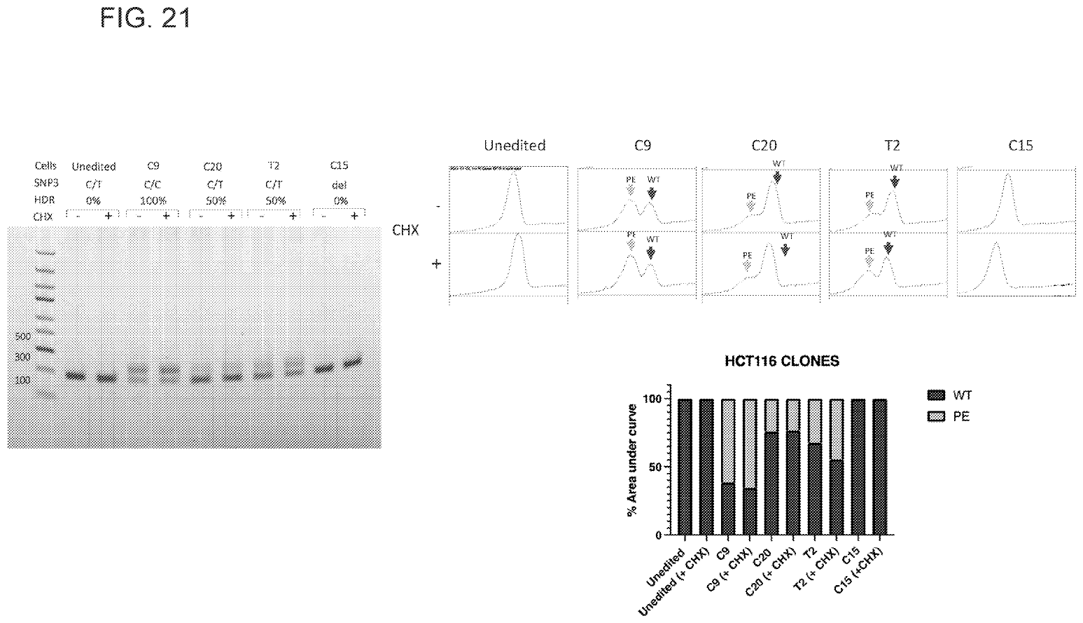

The results are shown in FIG. 21. As shown in FIG. 21, PCR of HTT mRNA from edited cells resulted in a second, longer, product that included the poison exon, along with the normal HTT product found in both samples. In a clonal line selected for successful editing of the ‘C’ allele by the ‘C’ guide, the longer product was shown to be ˜25% of the total mRNA produced from the HTT gene, showing that the edited allele was spliced to the longer form at ˜50%.

In a clonal line selected for successful editing of the ‘T’ allele by the ‘T’ guide, the longer product was shown to be ˜45% of the total mRNA produced from the HTT gene when NMD was inhibited, showing that the edited allele was spliced to the longer form at ˜90%. With NMD active, the longer form was ˜35% of the total HTT mRNA, showing substantial degradation of the longer mRNA. The results show that the majority of mRNA from the edited locus was spliced to include the poison exon, and that this mRNA was indeed degraded by NMD, reducing total HTT mRNA as intended.

A schematic depiction of the use of an allele-specific CRISPR-Cas guide RNA and a prime editor to introduce a poison exon, or to introduce a mutation that gives rise to a poison exon, in a target nucleic acid is depicted in FIG. 24B.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of reducing the level of an RNA transcript from a target nucleic acid, the method comprising modifying the nucleotide sequence of the target nucleic acid such that a spliced mRNA product of the modified target nucleic acid comprises a stop codon that is not present in a spliced mRNA transcript of the unmodified target nucleic acid and wherein, as a result of the stop codon, the spliced mRNA product undergoes nonsense-mediated mRNA decay, thereby reducing the level of the RNA transcript.

2. The method of claim 1, wherein the spliced mRNA product of the modified target nucleic acid comprises a stop codon induced by a frameshift caused by the inclusion of an exon not present in the spliced mRNA of the unmodified target nucleic acid.

3. The method of claim 2, wherein said modifying comprises: (a) modifying the nucleotide sequence in the target nucleic acid to include a splice dinucleotide that is not present in the unmodified target nucleic acid, optionally wherein the splice dinucleotide is an AG dinucleotide or a GT dinucleotide, (b) modifying a polypyrimidine tract that is immediately 5′ of an endogenous AG dinucleotide, generating a modified AG dinucleotide-containing sequence, such that a spliceosome in a cell would recognize the modified AG dinucleotide-containing sequence as a splice acceptor dinucleotide, and/or (c) modifying a consensus sequence within 10 nucleotides of an endogenous GT dinucleotide, generating a modified GT dinucleotide-containing sequence, such that a spliceosome in a cell would recognize the modified GT dinucleotide-containing sequence as a splice donor dinucleotide.

4-5. (canceled)

6. The method of claim 1, comprising modifying an exon of the target nucleic acid such that the modified target nucleic acid includes a stop codon that is not present in an exon of the unmodified target nucleic acid.

7. The method of claim 1, wherein modifying comprises contacting the target nucleic acid with a) a CRISPR-Cas effector polypeptide or a fusion CRISPR-Cas effector polypeptide comprising: i) a CRISPR-Cas effector polypeptide; and ii) one or more heterologous polypeptides; and (b) a guide nucleic acid, wherein the guide nucleic acid comprises: i) a targeting region that comprises a nucleotide sequence that binds to a target sequence in the target nucleic acid; and ii) a protein-binding region that binds to the CRISPR-Cas effector polypeptide.

8. The method of claim 7, further comprising contacting the target nucleic acid with a donor nucleic acid that comprises a nucleotide sequence that includes the modification that results in the stop codon.

9-10. (canceled)

11. The method of claim 7, wherein the one or more heterologous polypeptides comprises a reverse transcriptase, a cytidine deaminase, or an adenine deaminase.

12. The method of claim 11, wherein the CRISPR-Cas effector polypeptide is a nickase.

13-15. (canceled)

16. The method of claim 1, wherein the target nucleic acid is present in a eukaryotic cell.

17. The method of claim 16, wherein the eukaryotic cell is in vitro.

18. The method of claim 16, wherein the eukaryotic cell is in vivo.

19. The method of claim 16, wherein the eukaryotic cell comprises a disease-associated mutation-containing allele and a corresponding wild-type allele that does not comprise the disease-associated mutation, and wherein the disease-associated mutation-containing allele comprises the target nucleic acid.

20. The method of claim 19, wherein the disease-associated mutation-containing allele comprises a single nucleotide polymorphism (SNP) that is not present in the corresponding wild-type allele, and wherein the method comprises contacting the target nucleic acid with: a) a CRISPR-Cas effector polypeptide or a fusion polypeptide comprising a CRISPR-Cas effector polypeptide; and b) a guide nucleic acid, wherein the guide nucleic acid comprises: i) a targeting region that comprises a nucleotide sequence that binds to a target sequence in the target nucleic acid, wherein the target sequence comprises the SNP; and ii) a protein-binding region that binds to the CRISPR-Cas effector polypeptide.

21. The method of claim 1, wherein the target nucleic acid encodes a toxic gain-of-function polypeptide.

22. (canceled)

23. A method for treating a disease caused by a toxic gain-of-function polypeptide in an individual, the method comprising reducing the level of an RNA transcript from a target nucleic acid encoding the toxic gain-of-function polypeptide, comprising modifying the nucleotide sequence of the target nucleic acid such that a spliced mRNA product of the modified target nucleic acid comprises an exon that comprises a stop codon not present in a spliced mRNA product of the unmodified target nucleic acid, wherein the spliced mRNA product undergoes nonsense-mediated mRNA decay, thereby reducing the level of the RNA transcript and treating the disease.

24. The method of claim 23, wherein the disease is a trinucleotide repeat expansion disease, a tetranucleotide repeat expansion disease, or a hexanucleotide repeat expansion disease.

25-26. (canceled)

27. A system for reducing the level of an RNA transcript from a target nucleic acid in a eukaryotic cell, the system comprising:
a) a CRISPR-Cas effector polypeptide, or a nucleic acid encoding a CRISPR-Cas effector polypeptide, or a CRISPR-Cas effector fusion polypeptide, or a nucleic acid encoding a CRISPR-Cas effector fusion polypeptide, wherein the CRISPR-Cas effector fusion polypeptide comprises: i) the CRISPR-Cas effector polypeptide; and ii) one or more heterologous fusion partners;
b) a guide RNA comprising i) a targeting region that comprises a nucleotide sequence that binds to a target sequence in the target nucleic acid; and ii) a protein-binding region that binds to the CRISPR-Cas effector polypeptide; and
c) a donor nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′:
i) a nucleotide sequence of from about 5 nucleotides to about 15 nucleotides in length and having a first consensus splice motif;
ii) a nucleotide sequence of from about 11 nucleotides to about 15 nucleotides in length and comprising a stop codon in each reading frame; and
iii) a nucleotide sequence of from about 5 nucleotides to about 15 nucleotides in length and having a second consensus splice motif.

28-29. (canceled)

30. The system of claim 27, wherein the one or more heterologous polypeptides comprises a reverse transcriptase and wherein the guide RNA comprises a primer binding nucleotide sequence, and optionally wherein the CRISPR-Cas effector polypeptide is a nickase.

31-32. (canceled)

33. A composition comprising:
a) a CRISPR-Cas effector polypeptide, or a nucleic acid encoding a CRISPR-Cas effector polypeptide, or a CRISPR-Cas effector fusion polypeptide, or a nucleic acid encoding a CRISPR-Cas effector fusion polypeptide, wherein the CRISPR-Cas effector fusion polypeptide comprises: i) the CRISPR-Cas effector polypeptide; and ii) one or more heterologous fusion partners;
b) a guide RNA comprising i) a targeting region that comprises a nucleotide sequence that binds to a target sequence in the target nucleic acid; and ii) a protein-binding region that binds to the CRISPR-Cas effector polypeptide; and
c) a donor nucleic acid comprising a poison exon insertion nucleotide sequence that provides for insertion of a poison exon into the target nucleic acid, wherein the poison exon insertion nucleotide sequence has a length of from about 21 nucleotides to about 150 nucleotides and comprises, from 5′ to 3′:
i) a nucleotide sequence of from about 5 nucleotides to about 15 nucleotides in length and having a first consensus splice motif;
ii) a nucleotide sequence of from about 11 nucleotides to about 15 nucleotides in length and comprising a stop codon in each reading frame; and
iii) a nucleotide sequence of from about 5 nucleotides to about 15 nucleotides in length and having a second consensus splice motif.

34. (canceled)

35. The composition of claim 33, wherein:
the one or more heterologous polypeptides comprises a reverse transcriptase and wherein the guide RNA comprises a primer binding nucleotide sequence; or
the one or more heterologous polypeptides comprises a cytidine deaminase or an adenosine deaminase.

36-37. (canceled)


SEQUENCE LISTING (92 sequences)

SEQ ID NO: 1 - DNA, 45 bp, Homo sapiens
  source 1..45: mol_type = genomic DNA
ctagagactttttctggtgatgacaatttattaatagtcactttt

SEQ ID NO: 2 - DNA, 45 bp, synthetic construct
  source 1..45: mol_type = other DNA
ctagagactttttctggtgatggcaatttattaatagtcactttt

SEQ ID NO: 3 - DNA, 45 bp, synthetic construct
  source 1..45: mol_type = other DNA
ctaggggctttttctggtgatggcaatttattaatagtcactttt

SEQ ID NO: 4 - DNA, 58 bp, Homo sapiens
  source 1..58: mol_type = genomic DNA
ttgttctctctttttctttgggtgagagggtacacttgtgtttttgaatttatatgag

SEQ ID NO: 5 - RNA, 23 nt, synthetic construct
  source 1..23: mol_type = other RNA
acccaaagaaaaagagagaacaa

SEQ ID NO: 6 - DNA, 58 bp, synthetic construct
  source 1..58: mol_type = other DNA
ttgttctctctttttctttaggtgagagggtacacttgtgtttttgaatttatatgag

SEQ ID NO: 7 - DNA, 73 bp, synthetic construct
  source 1..73: mol_type = other DNA
ttgttctctctttttctttaggtgagagggtacacttgtgtttttgaatttatatgaggtaagtgtgtaatag

SEQ ID NO: 8 - DNA, 11 bp, synthetic construct
  source 1..11: mol_type = other DNA
tgactgactga

SEQ ID NO: 9 - protein, 1368 aa, Streptococcus pyogenes [sequence omitted]

SEQ ID NO: 10 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 11 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 12 - protein, 1306 aa, Acidaminococcus sp.
  source 1..1306: note = strain: BVL36
TQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 13 - protein, 1228 aa, Lachnospiraceae bacterium
  source 1..1228: note = strain: ND2006
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 14 - protein, 1129 aa, Alicyclobacillus acidoterrestris
MAVKSIKVKLRLDDMPEIRAGLWKLHKEVNAGVRYYTEWLSLLRQENLYRRSPNGDGEQECDKTAEECKAELLERLRARQVENGHRGPAGSDDELLQLARQLYELLVPQAIGAKGDAQQIARKFLSPLADKDAVGGLGIAKAGNKPRWVRMREAGEPGWEEEKEKAETRKSADRTADVLRALADFGLKPLMRVYTDSEMSSVEWKPLRKGQAVRTWDRDMFQQAIERMMSWESWNQRVGQEYAKLVEQKNRFEQKNFVGQEHLVHLVNQLQQDMKEASPGLESKEQTAHYVTGRALRGSDKVFEKWGKLAPDAPFDLYDAEIKNVQRRNTRRFGSHDLFAKLAEPEYQALWREDASFLTRYAVYNSILRKLNHAKMFATFTLPDATAHPIWTRFDKLGGNLHQYTFLFNEFGERRHAIRFHKLLKVENGVAREVDDVTVPISMSEQLDNLLPRDPNEPIALYFRDYGAEQHFTGEFGGAKIQCRRDQLAHMHRRRGARDVYLNVSVRVQSQSEARGERRPPYAAVFRLVGDNHRAFVHFDKLSDYLAEHPDDGKLGSEGLLSGLRVMSVDLGLRTSASISVFRVARKDELKPNSKGRVPFFFPIKGNDNLVAVHERSQLLKLPGETESKDLRAIREERQRTLRQLRTQLAYLRLLVRCGSEDVGRRERSWAKLIEQPVDAANHMTPDWREAFENELQKLKSLHGICSDKEWMDAVYESVRRVWRHMGKQVRDWRKDVRSGERPKIRGYAKDVVGGNSIEQIEYLERQYKFLKSWSFFGKVSGQVIRAEKGSRFAITLREHIDHAKEDRLKKLADRIIMEALGYVYALDERGKGKWVAKYPPCQLILLEELSEYQFNNDRPPSENNQLMQWSHRGVFQELINQAQVHDLLVGTMYAAFSSRFDARTGAPGIRCRRVPARCTQEHNPEPFPWWLNKFVVEHTLDACPLRADDLIPTGEGEIFVSPFSAEEGDFHQIHADLNAAQNLQQRLWSDFDISQIRLRCDWGEVDGELVLIPRLTGKRTADSYSNKVFYTNTGVTYYERERGKKRRKVFAQEKLSEEEAELLVEADEAREKSVVLMRDPSGIINRGNWTRQKEFWSMVNQRIEGYLVKQIRSRVPLQDSACENTGDI

SEQ ID NO: 15 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 16 - protein, 1601 aa, Desulfonema ishimotonii
MTTTMKISIEFLEPFRMTKWQESTRRNKNNKEFVRGQAFARWHRNKKDNTKGRPYITGTLLRSAVIRSAENLLTLSDGKISEKTCCPGKFDTEDKDRLLQLRQRSTLRWTDKNPCPDNAETYCPFCELLGRSGNDGKKAEKKDWRFRIHFGNLSLPGKPDFDGPKAIGSQRVLNRVDFKSGKAHDFFKAYEVDHTRFPRFEGEITIDNKVSAEARKLLCDSLKFTDRLCGALCVIRFDEYTPAADSGKQTENVQAEPNANLAEKTAEQIISILDDNKKTEYTRLLADAIRSLRRSSKLVAGLPKDHDGKDDHYLWDIGKKKKDENSVTIRQILTTSADTKELKNAGKWREFCEKLGEALYLKSKDMSGGLKITRRILGDAEFHGKPDRLEKSRSVSIGSVLKETVVCGELVAKTPFFFGAIDEDAKQTDLQVLLTPDNKYRLPRSAVRGILRRDLQTYFDSPCNAELGGRPCMCKTCRIMRGITVMDARSEYNAPPEIRHRTRINPFTGTVAEGALFNMEVAPEGIVFPFQLRYRGSEDGLPDALKTVLKWWAEGQAFMSGAASTGKGRFRMENAKYETLDLSDENQRNDYLKNWGWRDEKGLEELKKRLNSGLPEPGNYRDPKWHEINVSIEMASPFINGDPIRAAVDKRGTDVVTFVKYKAEGEEAKPVCAYKAESFRGVIRSAVARIHMEDGVPLTELTHSDCECLLCQIFGSEYEAGKIRFEDLVFESDPEPVTFDHVAIDRFTGGAADKKKFDDSPLPGSPARPLMLKGSFWIRRDVLEDEEYCKALGKALADVNNGLYPLGGKSAIGYGQVKSLGIKGDDKRISRLMNPAFDETDVAVPEKPKTDAEVRIEAEKVYYPHYFVEPHKKVEREEKPCGHQKFHEGRLTGKIRCKLITKTPLIVPDTSNDDFFRPADKEARKEKDEYHKSYAFFRLHKQIMIPGSELRGMVSSVYETVTNSCFRIFDETKRLSWRMDADHQNVLQDFLPGRVTADGKHIQKFSETARVPFYDKTQKHFDILDEQEIAGEKPVRMWVKRFIKRLSLVDPAKHPQKKQDNKWKRRKEGIATFIEQKNGSYYFNVVTNNGCTSFHLWHKPDNFDQEKLEGIQNGEKLDCWVRDSRYQKAFQEIPENDPDGWECKEGYLHVVGPSKVEFSDKKGDVINNFQGTLPSVPNDWKTIRTNDFKNRKRKNEPVFCCEDDKGNYYTMAKYCETFFFDLKENEEYEIPEKARIKYKELLRVYNNNPQAVPESVFQSRVARENVEKLKSGDLVYFKHNEKYVEDIVPVRISRTVDDRMIGKRMSADLRPCHGDWVEDGDLSALNAYPEKRLLLRHPKGLCPACRLFGTGSYKGRVRFGFASLENDPEWLIPGKNPGDPFHGGPVMLSLLERPRPTWSIPGSDNKFKVPGRKFYVHHHAWKTIKDGNHPTTGKAIEQSPNNRTVEALAGGNSFSFEIAFENLKEWELGLLIHSLQLEKGLAHKLGMAKSMGFGSVEIDVESVRLRKDWKQWRNGNSEIPNWLGKGFAKLKEWFRDELDFIENLKKLLWFPEGDQAPRVCYPMLRKKDDPNGNSGYEELKDGEFKKEDRQKKLTTPWTPWA

SEQ ID NO: 17 - protein, 966 aa, synthetic construct
IEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGNS

SEQ ID NO: 18 - protein, 1082 aa, Prevotella pectinovora
NQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMK

SEQ ID NO: 19 - protein, 1320 aa, Prevotella ihumii
  source 1..1320: note = sp. nov.
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSKFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKFKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSNSGTQFGGYLFRKKNEIGEYDYFLGISSKTQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIGNLLKTISCLKNKEEFHDLIKKDYQIFTEVQAVIDEICKQKTFIYFPVSNVELEKEMGDKDKPLCLFQISNKDLSFAKTFSANLRKKRGAENLHTMLFKALMSGNQDNLDLGSGAIFYRAKSLDGNKPTHPANEAIKCRNVANKDKVSLFTYDIYKNRRYMENKFLFHLSIVQNYKAAKDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGEMFVTRGQKIEKAVYQQFEKSLVDKLSYLVDKKRPYNELGGILKAYQLASSITKSNSDKQNGFLFYVPAWNTSKIDPVTGFTDLLRPKAMTIKEAQDFFGAFDNISYNDKGYFEFETNYDKFKIRMKGAQTRWTICTFGNRIKHKKDKNYWNYEEVELTEEFKKLFDKYDIDYRDGNLKEEILKIDNRKFFDALIKLLQLTLQLRNSDDKGNDYIVSPIANADGKFFNSNDGCKELPLDADANGAYNIARKGLWVVRQIKDKKDKISKLSNQEWLKFAQEKPYLK

SEQ ID NO: 20 - protein, 1323 aa, Prevotella disiens
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 21 - protein, 671 aa, Moloney murine leukemia virus
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 22 - skipped

SEQ ID NO: 23 - protein, 167 aa, unidentified
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 24 - protein, 178 aa, unidentified
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 25 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 26 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 27 - protein, 183 aa, Salmonella typhimurium
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 28 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 29 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 30 - protein, 161 aa, Caulobacter crescentus
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 31 - protein, 179 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 32 - protein, 198 aa, synthetic construct [sequence omitted]

SEQ ID NO: 33 - protein, 188 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 34 - protein, 7 aa, synthetic construct
PKKKRKV

SEQ ID NO: 35 - protein, 16 aa, synthetic construct
KRPAATKKAGQAKKKK

SEQ ID NO: 36 - protein, 9 aa, synthetic construct
PAAKRVKLD

SEQ ID NO: 37 - protein, 11 aa, synthetic construct
RQRRNELKRSP

SEQ ID NO: 38 - protein, 38 aa, synthetic construct
NQSSNFGPMKGGNFGGRSSGPYGGGGQYFAKPRNQGGY

SEQ ID NO: 39 - protein, 42 aa, synthetic construct
RMRIZFKNKGKDTAELRRRRVEVSVELRKAKKDEQILKRRNV

SEQ ID NO: 40 - protein, 8 aa, synthetic construct
VSRKRPRP

SEQ ID NO: 41 - protein, 8 aa, synthetic construct
PPKKARED

SEQ ID NO: 42 - protein, 8 aa, synthetic construct
PQPKKKPL

SEQ ID NO: 43 - protein, 12 aa, synthetic construct
SALIKKKKKMAP

SEQ ID NO: 44 - protein, 5 aa, synthetic construct
DRLRR

SEQ ID NO: 45 - protein, 7 aa, synthetic construct
PKQKKRK

SEQ ID NO: 46 - protein, 10 aa, synthetic construct
RKLKKKIKKL

SEQ ID NO: 47 - protein, 10 aa, synthetic construct
REKKKFLKRR

SEQ ID NO: 48 - protein, 20 aa, synthetic construct
KRKGDEVDGVDEVAKKKSKK

SEQ ID NO: 49 - protein, 17 aa, synthetic construct
RKCLQAGMNLEARKTKK

SEQ ID NO: 50 - protein, 11 aa, synthetic construct
YGRKKRRQRRR

SEQ ID NO: 51 - protein, 12 aa, synthetic construct
RRQRRTSKLMKR

SEQ ID NO: 52 - protein, 27 aa, synthetic construct
GWTLNSAGYLLGKINLKALAALAKKIL

SEQ ID NO: 53 - protein, 33 aa, synthetic construct
KALAWEAKLAKALAKALAKHLAKALAKALKCEA

SEQ ID NO: 54 - protein, 16 aa, synthetic construct
RQIKIWFQNRRMKWKK

SEQ ID NO: 55 - protein, 9 aa, synthetic construct
RKKRRQRRR

SEQ ID NO: 56 - protein, 8 aa, synthetic construct
RKKRRQRR

SEQ ID NO: 57 - protein, 11 aa, synthetic construct
YARAAARQARA

SEQ ID NO: 58 - protein, 11 aa, synthetic construct
THRLPRRRRRR

SEQ ID NO: 59 - protein, 11 aa, synthetic construct
GGRRARRRRRR

SEQ ID NO: 60 - protein, 20 aa, synthetic construct
  VARIANT 1..20: note = Wherein up to 9 copies of GS may be deleted
GSGSGSGSGSGSGSGSGSGS

SEQ ID NO: 61 - protein, 50 aa, synthetic construct
  VARIANT 1..50: note = Wherein up to 9 copies of GSGGS may be deleted
GSGGSGSGGSGSGGSGSGGSGSGGSGSGGSGSGGSGSGGSGSGGSGSGGS

SEQ ID NO: 62 - protein, 60 aa, synthetic construct
  VARIANT 1..60: note = Wherein up to 9 copies of GGSGGS may be deleted
GGSGGSGGSGGSGGSGGSGGSGGSGGSGGSGGSGGSGGSGGSGGSGGSGGSGGSGGSGGS

SEQ ID NO: 63 - protein, 50 aa, synthetic construct
  VARIANT 1..50: note = Wherein up to 9 copies of GGGGS may be deleted
GGGGSGGGGSGGGGSGGGGSGGGGSGGGGSGGGGSGGGGSGGGGSGGGGS

SEQ ID NO: 64 - protein, 4 aa, synthetic construct
GGSG

SEQ ID NO: 65 - protein, 5 aa, synthetic construct
GGSGG

SEQ ID NO: 66 - protein, 5 aa, synthetic construct
GSGSG

SEQ ID NO: 67 - protein, 5 aa, synthetic construct
GSGGG

SEQ ID NO: 68 - protein, 5 aa, synthetic construct
GGGSG

SEQ ID NO: 69 - protein, 5 aa, synthetic construct
GSSSG

SEQ ID NO: 70 - DNA, 79 bp, synthetic construct
  source 1..79: mol_type = other DNA
  misc_difference 10..34: note = Wherein up to 25 copies of n may be deleted
  misc_difference 46..70: note = Wherein up to 25 copies of n may be deleted
tttttcaggnnnnnnnnnnnnnnnnnnnnnnnnntgactaactagnnnnnnnnnnnnnnnnnnnnnnnnngtaagtatt

SEQ ID NO: 71 - RNA, 20 nt, synthetic construct
  source 1..20: mol_type = other RNA
gtcactccttccactatgtg

SEQ ID NO: 72 - RNA, 20 nt, synthetic construct
  source 1..20: mol_type = other RNA
gtcgctccttccactatgtg

SEQ ID NO: 73 - DNA, 100 bp, synthetic construct
  source 1..100: mol_type = other DNA
tgtacagttcacaaagcttaaaaaaatgctacctgccatttttttttcagcgaggaaggtgatacacagagagaccaagtgactgtgtccacggcgacgg

SEQ ID NO: 74 - DNA, 107 bp, Homo sapiens
  source 1..107: mol_type = genomic DNA
ggtgtacagttcacaaagcttaaaaaaatgctacctgccatttcatcctcagcgaggaaggtgatacacagagagaccaagtgactgtgtccacggcgacggcgctc

SEQ ID NO: 75 - RNA, 20 nt, synthetic construct
  source 1..20: mol_type = other RNA
ctcgctgaggatgaaatggc

SEQ ID NO: 76 - DNA, 100 bp, synthetic construct
  source 1..100: mol_type = other DNA
gtgtaatggtgtacagttcacaaagcttaaaaaaatgctatttgttatttcatcctcagcgaggaaggtgatacacagagagaccaagtgactgtgtcca

SEQ ID NO: 77 - DNA, 22 bp, synthetic construct
  source 1..22: mol_type = other DNA
ttctggccattttgagggttct

SEQ ID NO: 78 - DNA, 22 bp, synthetic construct
  source 1..22: mol_type = other DNA
tgctcactcatttccaccttca

SEQ ID NO: 79 - skipped

SEQ ID NO: 80 - DNA, 1551 bp, Homo sapiens
  source 1..1551: mol_type = genomic DNA
ttaatcttccttttttcttcagctgtgacttatgtattatgtttattttaggcgtccgtgagcactgttcaactgtggatatcgggaattctggccattttgagggttctgatttcccagtcaactgaagatattgttctttctcgtattcaggagctctccttctctccgtatttaatctcctgtacagtaattaataggttaagagatggggacagtacttcaacgctagaagaacacagtgaagggaaacaaataaagaatttgccagaagaaacattttcaaggtatgctttctatctgagcctataactaacccatgccttttgggaagtcacgtgatgtttcacagtcagtaagtctggaataatacctggtcttgcttcacttctgagttgggtaaagaagtctgtatcagtgtaattttctaatccgtcctgcattatctatggctcttggttcatacctgtcttgaagttctgtcatgttctgtctcttgtcctcagtagagatgctacagcagtggctcgcctcaggcagggcagggcagtggggtggctgtcctgggggcaggcagtaggggcacgctgacgtcagggaagttgaaacccaagagaagccagtaaaagtgagtctcagattgtcaccatgtgctggcagttttacacgctgtcagtaataaaagtcttctccctgcagggcagcctgcctccaataaatacgtgtagtatcaaatcctgtcttccctcataaattgtttggaagctccccaaggacagtgatgaggcactcgtaagtgcttgctgcctagatgggtccctctccacctttgctagattctgagcattcactgagttagagctgcttctgcaaatgtgctgcttctgctaagtggctgtgacttcatgcagccttcacttggtttgtcatcagtggagatgccctgtgttgtcgaaggagataagcccagtaagcctgctgggcaccttttggtttgcaggttcagcaggcagcccatggctttccctgtgtcgcattgaagcagctggctaaaattgatgatacattaaattcctgtgacagatgatcagcttgtatttgtgtaatggtgtacagttcacaaagcttaaaaaaatgctacctgccatttcatcctcagcgaggaaggtgatacacagagagaccaagtgactgtgtccacggcgacggcgctctgcatttcactttagcggttaatgtactctacctatatttttactttatatttaccatatatcttttcatgtatacttggcgtaagtgctttatagtagtcacctaattcactgtcatcttttttgtttcttggaaggtttctattacaactggttggtattcttttagaagacattgttacaaaacagctgaaggtggaaatgagtgagcagcaacatactttctattgccaggaactaggcacactgctaatgtgtctgatccacatcttcaagtctggtaggtgaatcacattagtcttcctggagtgtctcgttccccattctgcactataca

SEQ ID NO: 81 - DNA, 109 bp, Homo sapiens
  source 1..109: mol_type = genomic DNA
atgctacctgccatttcatcctcagcgaggaaggtgatacacagagagaccaagtgactgtgtccacggcgacggcgctctgcatttcactttagcggttaatgtactc

SEQ ID NO: 82 - DNA, 94 bp, Homo sapiens
  source 1..94: mol_type = genomic DNA
tacctgccatttcatcctcagcgaggaaggtgatacacagagagaccaagtgactgtgtccacggcgacggcgctctgcatttcactttagcgg

SEQ ID NO: 83 - DNA, 22 bp, synthetic construct
  source 1..22: mol_type = other DNA
cgaaggagataagcccagtaag

SEQ ID NO: 84 - DNA, 23 bp, synthetic construct
  source 1..23: mol_type = other DNA
gagacactccaggaagactaatg

SEQ ID NO: 85 - RNA, 20 nt, synthetic construct
  source 1..20: mol_type = other RNA
gtgtatcaccttcctcgctg

SEQ ID NO: 86 - RNA, 20 nt, synthetic construct
  source 1..20: mol_type = other RNA
gtgtatcaccttcctcactg

SEQ ID NO: 87 - DNA, 100 bp, synthetic construct
  source 1..100: mol_type = other DNA
tgtacagttcacaaagcttaaaaaaatgctacctgccatttttttttcagtgaggaaggtgatacacagagagaccaagtgactgtgtccacggcgacgg

SEQ ID NO: 88 - DNA, 100 bp, synthetic construct
  source 1..100: mol_type = other DNA
tggacacagtcacttggtctctctgtgtatcaccttcctcgctgaggatgaaataacaaatagcatttttttaagctttgtgaactgtacaccattacac

SEQ ID NO: 89 - DNA, 35 bp, synthetic construct
  source 1..35: mol_type = other DNA
gctcttccgatcttgaagcagctggctaaaattga

SEQ ID NO: 90 - DNA, 34 bp, synthetic construct
  source 1..34: mol_type = other DNA
gctcttccgatcttaaagtgaaatgcagagcgcc

SEQ ID NO: 91 - DNA, 22 bp, synthetic construct
  source 1..22: mol_type = other DNA
ttctggccattttgagggttct

SEQ ID NO: 92 - DNA, 22 bp, synthetic construct
  source 1..22: mol_type = other DNA
tgctcactcatttccaccttca